(19) United States
(12) Patent Application Publication
(10) Pub. No.: US 2021/0128226 A1
(43) Pub. Date: May 6, 2021

(54) TREATMENT INSTRUMENT AND TREATMENT SYSTEM

(71) Applicant: OLYMPUS CORPORATION, Tokyo (JP)

(72) Inventor: Tsunetaka AKAGANE, Hachioji-shi (JP)

(73) Assignee: OLYMPUS CORPORATION, Tokyo (JP)

(21) Appl. No.: 17/149,808

(22) Filed: Jan. 15, 2021

Related U.S. Application Data
(63) Continuation of application No. PCT/JP2018/026988, filed on Jul. 18, 2018.

(51) Int. Cl. A61B 18/14

(52) U.S. Cl. CPC A61B 18/1445; A61B 17/320092

(57) ABSTRACT

A treatment instrument includes first and second treatment bodies. The second treatment body includes a contact portion and a first surface. The first surface is adjacent to the contact portion in a width direction perpendicular to a longitudinal axis. The first surface includes a proximity edge which is in proximity to the contact portion; and an outer edge spaced from the contact portion from the longitudinal axis toward a side surface of a treatment portion in the width direction. Assuming that a virtual plane is defined to be perpendicular to the opening and closing directions in the closed state and passing through the proximity edge of the first surface, a distance between the outer edge and the virtual plane is larger than a distance between the proximity edge and the virtual plane.

### CROSS-REFERENCE TO RELATED APPLICATIONS

This application is a Continuation Application of PCT Application No. PCT/JP2018/026988, filed Jul. 18, 2018, the entire contents of which are incorporated herein by reference.

### FIELD

The present invention relates generally to a treatment instrument and a treatment system configured to perform an appropriate treatment on a treatment target.

### BACKGROUND

For example, Jpn. Pat. Appln. KOKAI Publication No. 2017-225882 discloses a treatment instrument that includes a treatment portion including a heater and a pair of high-frequency electrodes. The treatment portion includes a pair of grasping pieces, one of them, namely, a first grasping piece, is provided with the heater together with a first electrode. The other, namely, a second grasping piece is provided with a second electrode. In a state in which a treatment target is grasped by the treatment portion, the second grasping piece, in cooperation with the first grasping piece, applies a grasping pressure to the treatment target from the center to the outer edge of the second electrode in the width direction.

When a high-frequency current is caused to flow to the treatment target between the first electrode and the second electrode, the treatment target is coagulated. When the heater of the first grasping piece is heated and the heat is transferred to the treatment target, the treatment target is incised. When the latter treatment is performed, a high-frequency current may be caused to flow to the treatment target between the first electrode and the second electrode.

### SUMMARY

According to one aspect of the invention, a treatment instrument includes: a first treatment body having a treatment surface to be used as a high-frequency electrode, and configured to receive, together or separately, a high-frequency energy and another energy different from the high-frequency energy input to the treatment surface; and a second treatment body configured to treat a treatment target in cooperation with the treatment surface. The second treatment body includes: a contact portion extending along a longitudinal axis and having an electrical insulating property; and a first surface used as a first high-frequency electrode. The first surface is adjacent to the contact portion in a first width direction perpendicular to the longitudinal axis. The contact portion faces the treatment surface of the first treatment body. The contact portion is configured to move between an opened state in which the contact portion is separated from the treatment surface along opening and closing directions and a closed state in which the contact portion is closed to the treatment surface. The contact portion is configured to be brought into contact with the treatment surface in the closed state. The first surface is separated from the treatment surface in the opened state and the closed state, and includes a first proximity edge of the first surface, the first proximity edge being in proximity to the contact portion; and a first outer edge spaced from the contact portion from the longitudinal axis toward a first side surface of a treatment portion in the first width direction. Assuming that a virtual plane is defined to be perpendicular to the opening and closing directions in the closed state and passing through the first proximity edge of the first surface, in a distance between the virtual plane and the first surface, a distance between the first outer edge and the virtual plane is larger than a distance between the first proximity edge and the virtual plane.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing a treatment system in which a treatment instrument according to an exemplary embodiment is used.

FIG. 2 is a cross-sectional view schematically showing a state in which a treatment portion of the treatment instrument according to an exemplary embodiment is closed, in a cross section substantially perpendicular to an extending direction of the treatment portion.

FIG. 3A is a cross-sectional view schematically showing a state in which a treatment portion of the treatment instrument according to an exemplary embodiment is opened, in a cross section substantially perpendicular to the extending direction of the treatment portion.

FIG. 3B is a schematic enlarged view of a position indicated by a reference numeral 3B in FIG. 3A.

FIG. 4 is a cross-sectional view schematically showing a state in which a treatment target is grasped between a first grasping piece and a second grasping piece of the treatment portion of the treatment instrument according to the first embodiment, in a cross section substantially perpendicular to the extending direction of the treatment portion.

FIG. 5A is a cross-sectional view schematically showing a state in which a treatment target is grasped between a first grasping piece and a second grasping piece of a treatment portion of a treatment instrument according to a first modification of an exemplary embodiment, in a cross section substantially perpendicular to an extending direction of the treatment portion.

FIG. 5B is a schematic enlarged view of a position indicated by a reference numeral 5B in FIG. 5A.

FIG. 6A is a cross-sectional view schematically showing a state in which a treatment target is grasped between a first grasping piece and a second grasping piece of a treatment portion of a treatment instrument according to an exemplary embodiment, in a cross section substantially perpendicular to an extending direction of the treatment portion.

FIG. 6B is a cross-sectional view schematically showing a state in which a treatment target is grasped between a first grasping piece and a second grasping piece of a treatment portion of a treatment instrument according to an exemplary embodiment, in a cross section substantially perpendicular to an extending direction of the treatment portion.

FIG. 7 is a cross-sectional view schematically showing a state in which a treatment target is grasped between a first grasping piece and a second grasping piece of a treatment portion of a treatment instrument according to of an exemplary embodiment, in a cross section substantially perpendicular to an extending direction of the treatment portion.

FIG. 8 is a cross-sectional view schematically showing a state in which a treatment target is grasped between a first grasping piece and a second grasping piece of a treatment portion of a treatment instrument according to an exemplary embodiment, in a cross section substantially perpendicular to an extending direction of the treatment portion.

FIG. 9 is a cross-sectional view schematically showing a state in which a treatment target is grasped between a first grasping piece and a second grasping piece of a treatment portion of a treatment instrument according to an exemplary embodiment, in a cross section substantially perpendicular to an extending direction of the treatment portion.

FIG. 10 is a schematic view showing a treatment system in which a treatment instrument according to an exemplary embodiment is used.

FIG. 11 is a schematic view showing a configuration of a distal end portion of a shaft and a treatment portion of the treatment instrument according to an exemplary embodiment as viewed from one side in a width direction of the treatment portion, and a part thereof is shown in a cross section perpendicular or substantially perpendicular to the width direction of the treatment portion.

FIG. 12 is a schematic view showing a configuration of the distal end portion of the shaft and the treatment portion of the treatment instrument according an exemplary embodiment, as viewed from one side in a direction parallel or substantially parallel to a rotation axis of the treatment portion, and also showing an internal configuration of the shaft.

FIG. 13 is a cross-sectional view schematically showing a state in which a treatment portion of the treatment instrument according to an exemplary embodiment is closed, in a cross section substantially perpendicular to an extending direction of the treatment portion (a cross section taken along line XIII-XIII in FIG. 12).

FIG. 14 is a cross-sectional view schematically showing a state in which a treatment portion of the treatment instrument according to an exemplary embodiment is opened, in a cross section substantially perpendicular to the extending direction of the treatment portion.

FIG. 15 is a cross-sectional view schematically showing a state in which a treatment target is grasped between a first grasping piece and a second grasping piece of the treatment portion of the treatment instrument according to an exemplary embodiment, in a cross section substantially perpendicular to the extending direction of the treatment portion.

FIG. 16 is a cross-sectional view schematically showing a state in which a treatment target is grasped between a first grasping piece and a second grasping piece of a treatment portion of a treatment instrument according to an exemplary embodiment, in a cross section substantially perpendicular to an extending direction of the treatment portion.

FIG. 17 is a cross-sectional view schematically showing a state in which a treatment target is grasped between a first grasping piece and a second grasping piece of a treatment portion of a treatment instrument according to an exemplary embodiment, in a cross section substantially perpendicular to an extending direction of the treatment portion.

### DETAILED DESCRIPTION

Hereinafter, embodiments of the present invention will be described with reference to the drawings.

### First Embodiment

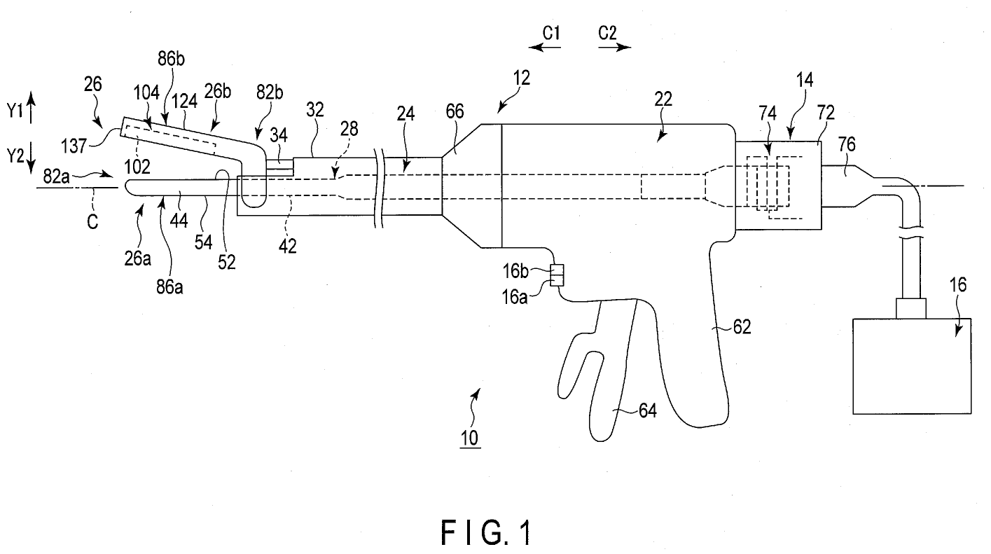

A first embodiment will be described with reference to FIG. 1 to FIG. 4. In the following, a treatment instrument 12 that performs a treatment on a living tissue using ultrasonic vibrations and a high-frequency current will be described. In the present embodiment, a high-frequency energy (high-frequency power) is applied to a blade 44 of a treatment portion 26, which will be described later, or ultrasonic vibrations are input as another energy (second energy) different from the high-frequency energy, separately from the application of the high-frequency energy.

A treatment system 10 includes the treatment instrument 12, a transducer unit 14 configured to generate ultrasonic vibrations, and an energy source 16.

The treatment instrument 12 includes a housing 22, a shaft 24, and a treatment portion (an end effector) 26. A rod 28 used as a part of the treatment portion 26 is inserted through the shaft 24. In the present embodiment, a longitudinal axis C is defined as a straight central axis with respect to the shaft 24 and the rod 28.

The treatment portion 26 includes a first grasping piece 26a and a second grasping piece 26b which can relatively approach and separate from each other. The first grasping piece 26a extends distally from the distal end of the shaft 24 along the longitudinal axis C as the central axis. The second grasping piece 26b is rotatably supported with respect to a distal end portion of the shaft 24. In the treatment portion 26, the second grasping piece 26b is rotated with respect to the first grasping piece 26a, thereby defining opening and closing directions relatively approaching and separating from each other. The opening and closing directions intersect the extending direction of the longitudinal axis C; for example, they are substantially perpendicular to an extending direction of the treatment portion 26 with respect to the distal end of the shaft 24.

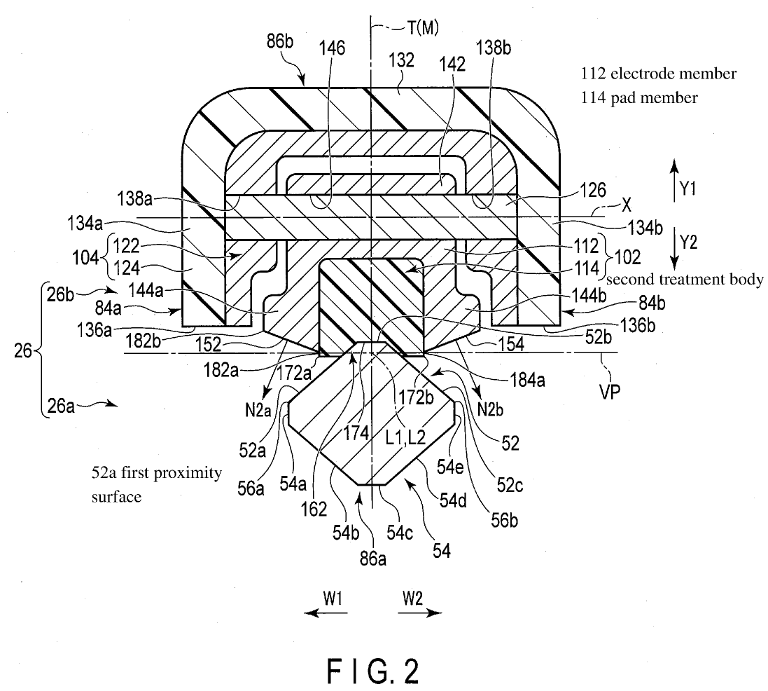

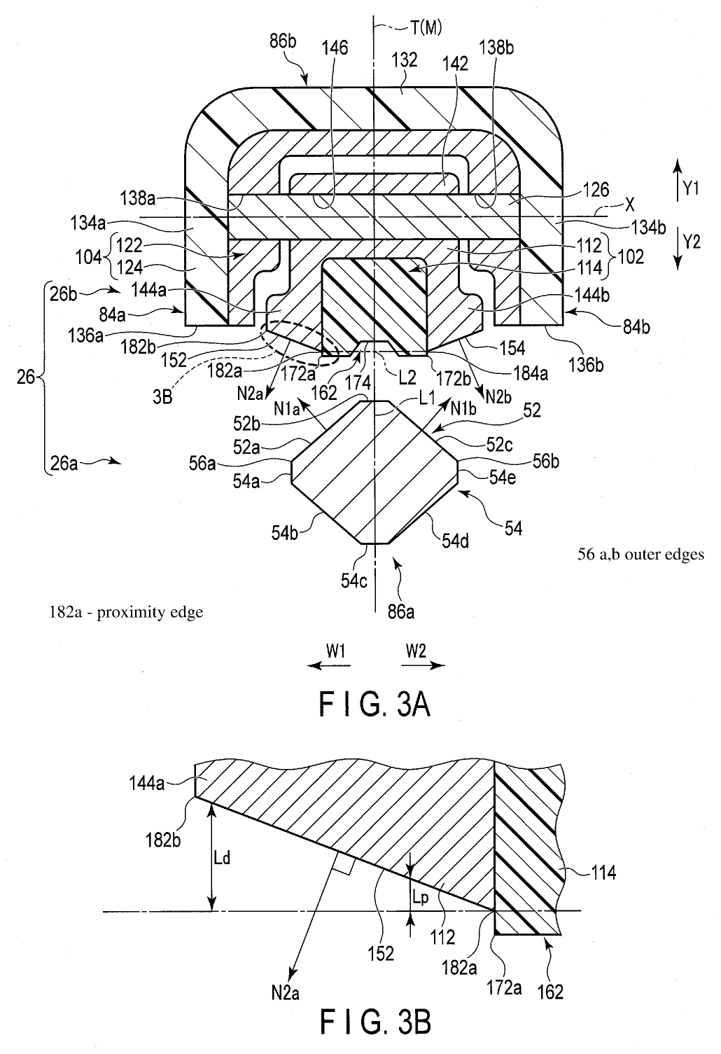

In the embodiment in which the extending direction of the treatment portion 26 is substantially parallel to the longitudinal axis C, the cross sections of FIG. 2 to FIG. 3B are cross sections substantially perpendicular to the longitudinal direction along the longitudinal axis C. FIG. 2 shows a closed state in which no treatment target is located between the first grasping piece 26a and the second grasping piece 26b and the second grasping piece 26b is closed with respect to the first grasping piece 26a. FIG. 3A shows an opened state in which no treatment target is located between the first grasping piece 26a and the second grasping piece 26b and the second grasping piece 26b is opened with respect to the first grasping piece 26a. Here, a direction intersecting (substantially perpendicular to) the extending direction of the treatment portion 26 and intersecting (substantially perpendicular to) the opening and closing directions of the second grasping piece 26b is defined as width directions of the treatment portion 26 (a direction indicated by an arrow W1 and a direction indicated by an arrow W2). The second width direction W2 is opposite to the first width direction W1.

A longitudinal axis L1 is defined in the first grasping piece 26a. A longitudinal axis L2 is defined in the second grasping piece 26b. The longitudinal axis L1 passes through a central position M in the width direction of the first grasping piece 26a. The longitudinal axis L2 passes through a central position M in the width direction of the second grasping piece 26b. In the opened state of the second grasping piece 26b relative to the first grasping piece 26a, the longitudinal axes L1 and L2 deviate from each other. The longitudinal axis L2 moves relative to the longitudinal axis C as the second grasping piece 26b rotates about the shaft 24. In the closed state of the second grasping piece 26b relative to the first grasping piece 26a shown in FIG. 2, the longitudinal axes L1 and L2 coincide.

The longitudinal axis L1 is virtual and may be either on a central surface 52b of a facing surface 52 of the blade 44 or between the facing surface 52 and a non-facing surface 54. Alternatively, the longitudinal axis L1 may be outside the blade 44.

The longitudinal axis L2 is virtual, and may be either on a contact portion 162 of a pad member 114 of a blade 102 to be described later or outside the contact portion 162 of the pad member 114. Alternatively, the longitudinal axis L2 may be inside the contact portion 162 of the pad member 114.

The opening and closing directions of the first grasping piece 26a and the second grasping piece 26b are along a virtual motion surface T defined by the first grasping piece 26a and the second grasping piece 26b. The motion surface T is preferably planar. The motion surface T is substantially parallel to the extending direction of the treatment portion 26 and substantially parallel to the opening and closing directions of the second grasping piece 26b.

In the present embodiment, the first grasping piece 26a and the second grasping piece 26b of the treatment portion 26 are symmetrical with respect to the motion surface T. At this time, the longitudinal axes L1 and L2 are on the motion surface T.

The width direction of the treatment portion 26 intersects (is substantially perpendicular to) the motion surface T. In the present embodiment, for example, the motion surface T passes through the central position M in the width direction of the second grasping piece 26b over the entire range from the proximal end to the distal end of the second grasping piece 26b. Thus, the motion surface T is a center plane of the second grasping piece 26b. Since the motion surface T is defined as described above, the motion surface T passes through the treatment portion 26.

The shaft 24 is formed of an electrical conductive material. The outer peripheral surface of the shaft 24 is coated with an electrical insulating material, such as PTFE. The rod 28 is made of a material having good vibration transmissibility and having electrical conductivity, such as a titanium alloy material. For example, an electrical insulating spacer (not shown) is disposed between the inner peripheral surface of the shaft 24 and the outer peripheral surface of the rod 28. Therefore, the shaft 24 and the rod 28 are electrically insulated from each other, and an unintended current flow between the shaft 24 and the rod 28 is prevented.

The shaft 24 has a circular outer shape in a cross section perpendicular to the longitudinal axis C. The shaft 24 includes a pipe 32 and a movable member 34 configured to move relative to the pipe 32 along the longitudinal axis C.

The rod 28 includes a rod body 42 and the blade (first treatment body) 44 provided at a distal end portion of the rod body 42. The blade 44 serves as the first grasping piece 26a. The blade 44 distally protrudes from the distal end of the shaft 24 along the longitudinal axis L1.

The longitudinal axis L1 of the blade 44 of the first grasping piece 26a is, for example, parallel or substantially parallel to the longitudinal axis C of the shaft 24. In this case, the blade 44 extends substantially straight distally from the shaft 24. The longitudinal axis L1 of the distal end portion of the blade 44 may bend with respect to the longitudinal axis C of the shaft 24.

A cross section perpendicular to the longitudinal axis L1 of the blade 44 is polygonal or substantially polygonal. In the present embodiment, an example will be described in which the cross section perpendicular to the longitudinal axis L1 of the blade 44 is substantially octagonal.

The blade 44 includes the facing surface (treatment surface) 52, facing the blade (second treatment body) 102 (to be described later) of the second grasping piece 26b. The blade 44 is used as a high-frequency electrode, and the facing surface 52 is used as a treatment surface of the electrode (electrode surface). The facing surface 52 may be a flat surface or a curved surface. The facing surface 52 may be a combination of a plurality of curved surfaces and/or flat surfaces. In the present embodiment, the facing surface 52 includes three surfaces 52a, 52b, and 52c. The surface (first proximity surface) 52a is brought in proximity to a first side surface 84a (described later) of the treatment portion 26. The surface 52b is formed as a central surface in a central portion in the width direction of the facing surface 52. The surface (second proximity surface) 52c is brought in proximity to a second side surface 84b (described later) of the treatment portion 26.

The blade 44 of the first grasping piece 26a includes the non-facing surface 54, which does not face the blade 102 of the second grasping piece 26b. The non-facing surface 54 may be a flat surface or a curved surface. The non-facing surface 54 may be a combination of a plurality of curved surfaces and/or flat surfaces. In this embodiment, the non-facing surface 54 includes five flat surfaces 54a, 54b, 54c, 54d, and 54e. The surface 54a of the non-facing surface 54 is brought in proximity to the first side surface 84a of the treatment portion 26, and is adjacent to the first proximity surface 52a of the facing surface 52. The surface 54e of the non-facing surface 54 is brought in proximity to the second side surface 84b of the treatment portion 26, and is adjacent to the second proximity surface 52c of the facing surface 52. The surface 54c of the non-facing surface 54 is formed as a central surface in a central portion in the width direction of the non-facing surface 54, and is formed on the back of the central surface 52b of the facing surface 52. The surface 54c of the non-facing surface 54 is formed as a back surface 86a of the first grasping piece 26a.

The five surfaces 54a, 54b, 54c, 54d, and 54e of the non-facing surface 54 are preferably coated with electrical insulating and heat-resistant resin.

The blade 44 of the first grasping piece 26a includes a first outer edge 56a and a second outer edge 56b. In the present embodiment, the first outer edge 56a of the blade 44 is a boundary between the surface 52a of the facing surface 52 and the surface 54a of the non-facing surface 54. The second outer edge 56b of the blade 44 is a boundary between the surface 52c of the facing surface 52 and the surface 54e of the non-facing surface 54.

The first outer edge 56a is closer to the first side surface 84a of the treatment portion 26 than to the second side surface 84b of the treatment portion 26. Therefore, the first outer edge 56a of the blade 44 is brought in proximity to the first side surface 84a of the treatment portion 26 separated from the longitudinal axis L1 of the blade 44 in the first width direction W1 perpendicular to the opening and closing directions along the motion surface T.

The second outer edge 56b is closer to the second side surface 84b of the treatment portion 26 than to the first side surface 84a of the treatment portion 26. Therefore, the second outer edge 56b of the blade 44 is brought in proximity to the second side surface 84b of the treatment portion 26 separated from the longitudinal axis L1 of the blade 44 in the second width direction W2 perpendicular to the opening and closing directions along the motion surface T.

In the present embodiment, the central surface 52b of the facing surface 52 between the first outer edge 56a and the second outer edge 56b of the blade 44 is used as a protrusion that is brought into contact with the contact portion 162 (to be described later) of the blade 102 of the second grasping piece 26b in cooperation with a region of the surfaces 52a and 52c that are adjacent to the central surface 52b.

A normal vector N1a on the first proximity surface 52a of the facing surface 52 of the blade 44 is considered. Of the normal vector N1a of the first proximity surface 52a, components parallel to a virtual plane VP to be described later are directed from the first proximity surface 52a to the first side surface 84a of the treatment portion 26 at any position of the first proximity surface 52a. A normal vector N1b on the second proximity surface 52c of the facing surface 52 of the blade 44 is considered. Of the normal vector N1b of the second proximity surface 52c, components parallel to the virtual plane VP are directed from the second proximity surface 52c to the second side surface 84b of the treatment portion 26 at any position of the second proximity surface 52c.

The housing 22 includes a grip 62 extending in a direction intersecting the central axis C. A movable handle 64 is supported by the housing 22 on a distal side of the grip 62. The movable handle 64 is located on the side of the longitudinal axis C from which the grip 62 extends.

As the movable handle 64 rotates with respect to the housing 22, the movable handle 64 moves between an opened state and a closed state with respect to the grip 62. The moving direction in each of the opening operation and the closing operation of the movable handle 64 is substantially parallel to the longitudinal direction along the longitudinal axis C.

The movable handle 64 may be disposed on a proximal side of the grip 62. The movable handle 64 may be located on the opposite side of the longitudinal axis C from the side where the grip 62 is located. In this case, the moving direction in each of the opening operation and the closing operation of the movable handle 64 intersects the longitudinal direction.

The distal end portion of the movable member 34 of the shaft 24 supports the proximal end portion of the second grasping piece 26b. Although not shown, the proximal end portion of the movable member 34 is coupled to the movable handle 64 inside the housing 22.

The movable member 34 moves along the longitudinal axis C relative to the pipe 32 as the movable handle 64 separates from or approaches the grip 62. The second grasping piece 26b rotates relative to the first grasping piece 26a in accordance with the movement of the movable member 34. When the movable handle 64 is separated from the grip 62, the second grasping piece 26b is opened with respect to the first grasping piece 26a. When the movable handle 64 approaches the grip 62, the second grasping piece 26b is closed with respect to the first grasping piece 26a.

A rotation knob 66 is attached to the distal side of the housing 22. The rotation knob 66 is rotatable about the longitudinal axis C relative to the housing 22. The proximal end of the shaft 24 is inserted into the housing 22 from the distal side of the housing 22 through the inside of the rotation knob 66.

The distal end portion of the rod 28 extends distally from the distal end of the pipe 32 through the inside of the pipe 32 out of the housing 22.

A transducer unit 14 configured to generate ultrasonic vibrations is detachably coupled to the proximal side of the housing 22 of the treatment instrument 12. The transducer unit 14 includes a case 72 and a transducer 74 configured to generate longitudinal ultrasonic vibrations along the longitudinal axis C.

Inside the housing 22, the transducer 74 is connected to the proximal side of the rod 28. One end of a cable 76 is connected to the case 72. The other end of the cable 76 is connected to the energy source 16.

The energy source 16 in this embodiment outputs energy to the transducer 74 to cause the transducer 74 to generate ultrasonic vibrations. At this time, longitudinal ultrasonic vibrations are generated in the transducer 74. The longitudinal ultrasonic vibrations generated in the transducer 74 are input to the proximal end of the rod 28 and transmitted along the longitudinal axis C from the proximal end to the distal end of the rod 28. When the energy source 16 outputs energy for generating ultrasonic vibrations to the transducers 74, vibrations for cutting the treatment target are transmitted to the blade 44 of the first grasping piece 26a. In addition, the energy source 16 of the present embodiment is capable of supplying an electrical energy (high-frequency energy) through the shaft 24 and the rod 28 to the treatment target grasped between the first grasping piece 26a and the second grasping piece 26b of the treatment portion 26.

The energy source 16 supplies an electrical energy (high-frequency energy) to the treatment target grasped between the first grasping piece 26a and the second grasping piece 26b of the treatment portion 26 through the shaft 24 and the rod 28, for example, in response to pressing of a first switch 16a provided in the housing 22.

The energy source 16 outputs energy for generating ultrasonic vibrations to the transducers 74 from the energy source 16, for example, in response to pressing of a second switch 16b provided in the housing 22.

In one embodiment, the energy source 16 supplies an electrical energy (high-frequency energy) to the treatment target grasped between the first grasping piece 26a and the second grasping piece 26b of the treatment portion 26 through the shaft 24 and the rod 28 from the energy source, for example, in response to pressing of a second switch 16b provided in the housing 22, and outputs energy for generating ultrasonic vibrations to the transducers 74.

In the treatment portion 26 of the present embodiment, the dimension of the treatment portion 26 in the longitudinal direction is larger than each of the dimension of the treatment portion 26 in the opening and closing directions and the dimension of the treatment portion 26 in the width direction. The treatment portion 26, in which the first grasping piece 26a and the second grasping piece 26b cooperate, have a distal end portion 82a, a proximal end portion 82b, a first side surface (side portion) 84a, a second side surface (side portion) 84b, a first back surface 86a, and a second back surface 86b. In particular, the first back surface 86a is formed on the first grasping piece 26a and the second back surface 86b is formed on the second grasping piece 26b.

The first side surface 84a is separated from the longitudinal axes L1 and L2 in the first width direction W1. The second side surface 84b is separated from the longitudinal axes L1 and L2 in the second width direction W2 opposite to the first width direction W1.

The second grasping piece 26b includes the blade (second treatment body) 102 and a jaw (support body) 104 provided with the blade 102. Here, the second grasping piece 26b is described as a seesaw jaw or a wiper jaw in which the blade 102 is swingable with respect to the jaw 104. In one embodiment, the blade 102 is fixed relative to the jaw 104.

The blade 102 faces the facing surface 52 of the blade 44. The blade 102 includes an electrode member 112 and a pad member 114.

The electrode member 112 is formed of an electrical conductive material. The electrode member 112 is formed of, for example, an aluminum alloy or a metal containing aluminum. The pad member 114 is formed of an electrical insulating material. Since friction may occur between the pad member 114 and the blade 44 to which the longitudinal ultrasonic vibrations are transmitted, it is preferable to use a material having friction resistance and heat resistance to form the pad member 114. The pad member 114 is formed of, for example, polytetrafluoroethylene (PTFE) or the like.

The jaw 104 is supported, for example, on the pipe 32 of the shaft 24 and on the movable member 34. The jaw 104, together with the second grasping piece 26b, is movable to an opened state and a closed state relative to the blade 44 of the first grasping piece 26a, and is separated from the blade 44 of the first grasping piece 26a in both the opened state and the closed state.

The jaw 104 is rotatable about an attachment position of the shaft 24 to the distal end portion of the pipe 32 as a fulcrum. The jaw 104 includes a support member 122 formed of an electrical conductive material such as metal, and a cover 124 attached to an outer surface of the support member 122. The cover 124 is formed of an electrical insulating material such as a resin material. The distal end portion of the movable member 34 of the shaft 24 is connected to the support member 122. As described above, the movable member 34 is moved along the longitudinal axis C with respect to the pipe 32, whereby the jaw 104 and the blade 102 provided on the jaw 104 are rotated about the attachment position to the shaft 24, so that the second grasping piece 26b is opened or closed with respect to the first grasping piece 26a. A portion of the support member 122 exposed to the outside of the second grasping piece 26b is coated with a coating or the like having an electrical insulating property.

The jaw 104 includes a back wall 132, side walls 134a and 134b, edges 136a and 136b, and a distal end wall 137.

The back wall 132 forms the second back surface 86b of the treatment portion 26. The side wall 134a forms a portion of the first side surface 84a of the treatment portion 26 on the second grasping piece 26b side. The side wall 134b forms a portion of the second side surface 84b of the treatment portion 26 on the second grasping piece 26b side. The distal end wall 137 forms a portion of the distal end portion 82a of the treatment portion 26. The distal end wall 137 forms a distal end of the second grasping piece 26b, and forms a portion facing the distal side on the outer surface of the second grasping piece 26b.

Each of the back wall 132 and the side walls 134a and 134b extends from the distal end wall 137 toward the proximal side. In a cross section passing through the back wall 132 and the side walls 134a and 134b and substantially perpendicular to the extending direction of the second grasping piece 26b, the jaw 104 is substantially U-shaped. For this reason, the side walls 134a and 134b are separated from each other in the width direction. The back wall 132 forms an end of the second grasping piece 26b on a side (arrow Y1 side) where the second grasping piece 26b opens, that is, an end on a side opposite to a side where the first blade 44 is located. The back wall 132 forms a portion of the outer surface of the second grasping piece 26b facing the side where the second grasping piece 26b opens, that is, the back surface 86b of the second grasping piece 26b.

The side wall (first side wall) 134a forms one end of the second grasping piece 26b in the width direction. The side wall (second side wall) 134b forms the other end of the second grasping piece 26b in the width direction. The side wall 134a forms a portion facing one side in the width direction on the outer surface of the second grasping piece 26b, that is, one side surface of the second grasping piece 26b. The side wall (first side wall) 134a forms the first side surface 84a of the treatment portion 26.

The side wall 134b forms a portion facing the other side in the width direction on the outer surface of the second grasping piece 26b, that is, the other side surface of the second grasping piece 26b. The side wall (second side wall) 134b forms the second side surface 84b of the treatment portion 26.

The edges 136a and 136b form portions of the outer surfaces of the second grasping piece 26b facing the first grasping piece 26a. The edge 136a is adjacent to the side wall 134a. The edge 136b is adjacent to the side wall 134b.

In the distal end wall 137, the cover 124 is attached to the support member 122 from the distal side. In the back wall 132, the cover 124 is attached to the support member 122 from the side where the second grasping piece 26b opens. In each of the side walls 134a and 134b, the cover 124 is attached to the support member 122 from the outside in the width direction.

In the second grasping piece 26b, the electrode member 112 is attached to the jaw 104 via a connection pin 126. The electrode member 112 and the connection pin 126 are formed of an electrical conductive material such as metal. The electrode member 112 is provided on a side where the second grasping piece 26a is positioned with respect to the back wall 132 of the jaw 104, that is, on a side (arrow Y2 side) on which the first grasping piece 26b is closed. The electrode member 112 is provided on the inner side in the width direction with respect to the side walls 134a and 134b of the jaw 104. The electrode member 112 is disposed between the side walls 134a and 134b in the width direction.

The electrode member 112 includes a base 142 and side plates 144a and 144b. The back wall 132 of the jaw 104 is adjacent to the base 142 of the electrode member 112 on the side where the second grasping piece 26b opens. A clearance is formed between the base 142 and the back wall 132 in the opening and closing directions of the second grasping piece 26b. Each of the side plates 144a and 144b extends from the base 142 toward the side where the second grasping piece 26b is closed. In a cross section substantially perpendicular to the extending direction of the second grasping piece 26b, the electrode member 112 is formed in a substantially U-shape by the base 142 and the side plates 144a and 144b. For this reason, the side plates 144a and 144b are separated from each other in the width direction. The side wall 134a of the jaw 104 is adjacent to the side plate 144a of the electrode member 112 on the outer side in the width direction. The side wall 134b of the jaw 104 is adjacent to the side plate 144b of the electrode member 112 on the outer side in the width direction. A clearance is formed between the side plate 144a and the side wall 134a in the width direction, and a clearance is formed between the side plate 144b and the side wall 134b in the width direction.

A hole 146 penetrating the base 142 in the width direction is formed in the base 142 of the electrode member 112. A hole 138a is formed in the side wall 134a of the jaw 104 along the width direction. A hole 138b is formed in the side wall 134b of the jaw 104 along the width direction. The connection pin 126 that connects the jaw 104 (support member 122) and the electrode member 112 is inserted through the hole 146 and is inserted into each of the holes 138a and 138b. The connection pin 126 extends in the width direction through the holes 146 and the holes 138a and 138b. The electrode member 112 is swingable (rotatable) with respect to the jaw 104 about the central axis of the connection pin 126 as a swing axis X. In other words, the electrode member 112 swings about the swing axis X substantially parallel to the width direction.

When the electrode member 112 swings to one side about the swing shaft X, a portion of the electrode member 112 on the distal side with respect to the swing axis X approaches the first grasping piece 26a and moves away from the back wall 132 of the jaw 104. At this time, the portion of the electrode member 112 on the proximal side with respect to the swing axis X moves away from the first grasping piece 26a and approaches the back wall 132. Then, when the electrode member 112 abuts against the back wall 132 at a portion on the proximal side with respect to the swing axis X, the swing of the electrode member 112 to one side about the swing axis X is restricted. On the other hand, when the electrode member 112 swings to the other side about the swing shaft X, a portion of the electrode member 112 on the distal side with respect to the swing axis X moves away from the first grasping piece 26a and approaches the back wall 132 of the jaw 104. At this time, the portion of the electrode member 112 on the proximal side with respect to the swing axis X approaches the first grasping piece 26a and moves away from the back wall 132. Then, when the electrode member 112 abuts against the back wall 132 at a portion on the distal side with respect to the swing axis X, the swing of the electrode member 112 to the other side about the swing axis X is restricted.

A pair of electrode surfaces (inclined surfaces) 152 and 154, separated from each other, are formed on the outer surface of the electrode member 112. The electrode surfaces 152 and 154 face a side where the first grasping piece 26a is located, that is, a side where the second grasping piece 26b is closed. The first electrode surface 152 and the second electrode surface 154 extend, for example, in parallel with a longitudinal axis L2 of the contact portion 162 described later.

The first electrode surface 152 is adjacent to the inner sides of the side wall 134a and the edge 136a of the jaw 104 with a clearance in the width direction. In any state within the range in which the electrode member 112 is swingable, a part or the whole of the first electrode surface 152 protrudes from the edge 136a to the side where the first grasping piece 26a is located, that is, to the side where the second grasping piece 26b is closed. The first electrode surface 152 faces the first proximity surface 52a.

Similarly, the second electrode surface 154 is adjacent to the inner sides of the side wall 134b and the edge 136b of the jaw 104 with a clearance in the width direction. In any state within the range in which the electrode member 112 is swingable, a part or the whole of the second electrode surface 154 protrudes from the edge 136b to the side where the first grasping piece 26a is located, that is, to the side where the second grasping piece 26b is closed. The second electrode surface 154 faces the second proximity surface 52c.

The first electrode surface 152 is used as a treatment surface of a high-frequency electrode (first high-frequency electrode). The second electrode surface 154 is used as a treatment surface of a high-frequency electrode (second high-frequency electrode). The first electrode surface 152 and the second electrode surface 154 are electrically connected and have the same potential.

The first electrode surface (first surface) 152 is separated from the facing surface 52 of the blade 44 in both the opened state and the closed state. The second electrode surface (second surface) 154 is separated from the facing surface 52 of the blade 44 in both the opened state and the closed state.

In the second grasping piece 26b, the pad member 114 is fixed to the electrode member 112. In particular, the pad member 114 is fixed between the electrode surfaces 152 and 154 on the outer surfaces of the electrode member 112 in the width direction. The pad member 114 is swingable together with the electrode member 112 with respect to the jaw 104.

The pad member 114 is provided on a side of the base 142 of the electrode member 112 on which the second grasping piece 26a is located, that is, on a side where the first grasping piece 26b is closed. Furthermore, the pad member 114 is provided on the inner side of the side plates 144a and 144b of the electrode member 112 in the width direction, and is disposed between the side plates 144a and 144b in the width direction.

The pad member 114 includes the contact portion 162 that is located between the electrode surfaces 152 and 154, and faces the facing surface 52 of the blade (first treatment body) 44. Therefore, the blade 102 of the second grasping piece 26b includes the contact portion 162, the first electrode surface (first surface) 152, and the second electrode surface (second surface) 154 as the surfaces that face the facing surface 52 of the first grasping piece 26a.

The contact portion 162 has an electrical insulating property. The contact portion 162 faces the facing surface (treatment surface) 52 of the blade 44. The contact portion 162 is movable relative to the facing surface 52 of the blade 44 along the opening and closing directions between an opened state in which the contact portion is separated from the facing surface and a closed state in which the contact portion is brought in proximity to the facing surface 52. In particular, the contact portion 162 can be brought into contact with the facing surface 52 of the blade 44 in the closed state. That is, in the closed state, the central surface (protrusion) 52b of the facing surface 52 of the blade 44 of the first grasping piece 26a is also located between the electrode surfaces 152 and 154 in the width direction. Therefore, the central position M of the second grasping piece 26b in the width direction passes through the contact portion 162 of the pad member 114 and the central surface 52b of the facing surface 52 of the blade 44.

The contact portion 162 includes a first edge portion 172a separated from the central position M in the first width direction W1, a second edge portion 172b separated from the central position M in the second width direction W2, and a central portion 174 through which the central position M passes. The first edge portion 172a is closer to the first side surface 84a of the treatment portion 26 than to the second side surface 84b of the treatment portion 26. Therefore, the first edge portion 172a of the contact portion 162 is brought in proximity to the first side surface 84a of the treatment portion 26 which is separated from the longitudinal axis L2 of the contact portion 162 in the first width direction W1 perpendicular to the opening and closing directions. The second edge portion 172b is closer to the second side surface 84b of the treatment portion 26 than to the first side surface 84a of the treatment portion 26. Therefore, the second edge portion 172b of the contact portion 162 is brought in proximity to the second side surface 84b of the treatment portion 26 which is separated from the longitudinal axis L2 of the contact portion 162 in the second width direction W2 perpendicular to the opening and closing directions.

The central portion 174 is formed between the first edge portion 172a and the second edge portion 172b. Here, the central portion 174 is formed in a concave shape into which the vicinity of the central portion of the facing surface 52 of the blade 44 of the first grasping piece 26a is fitted. Therefore, in the present embodiment, the three surfaces 52a, 52b, and 52c of the facing surface 52 are brought into contact with the central portion 174 of the contact portion 162. Of course, only the central surface 52b may be in contact with the central portion 174 of the contact portion 162.

The first electrode surface 152 includes a first proximity edge 182a that is in proximity to the longitudinal axis L2 and a first outer edge 182b spaced farther from the longitudinal axis L2 than the first proximity edge 182a and in proximity to the first side surface 84a of the treatment portion 26.

It is preferable that there be no clearance between the first edge portion 172a of the contact portion 162 and the proximity edge 182a of the first electrode surface 152, but there may be a slight clearance between the first edge portion 172a and the proximity edge 182a. In addition, it is preferable that there be no step between the first edge portion 172a of the contact portion 162 and the proximity edge 182a of the first electrode surface 152, but there may be a slight step between the first edge portion 172a and the proximity edge 182a. Here, the contact portion 162 is at the same level as the first electrode surface 152 or protrudes toward the treatment surface 52 from the first electrode surface 152 when the second grasping piece 26b is closed with respect to the first grasping piece 26a.

The first electrode surface 152 is provided from the first edge portion 172a of the contact portion 162 or the first proximity edge 182a of the first electrode surface 152 toward the first side surface 84a of the treatment portion 26. In the present embodiment, the first electrode surface (first surface) 152 is formed in a flat shape between the first edge portion 172a of the contact portion 162 or the proximity edge 182a of the first electrode surface 152 and the outer edge 182b of the first electrode surface 152. Therefore, in one cross section, the portion between the proximity edge 182a of the first electrode surface 152 and the outer edge 182b of the first electrode surface 152 is linear.

The first electrode surface 152 is inclined in the width direction so as to separate from the side where the first grasping piece 26a is located as the distance to the first side surface 84a of the treatment portion 26 is reduced. In other words, the electrode surface 152 is directed toward the side where the second grasping piece 26b is opened as it separates from the central position M in the width direction.

Here, the contact portion 162 of the pad member 114 of the blade 102 of the second grasping piece 26b can be brought into contact with the facing surface 52 of the blade 44 of the first grasping piece 26a in the closed state. A virtual plane VP perpendicular to the opening and closing directions and passing through the first proximity edge 182a of the first electrode surface 152 in the closed state is defined on the first electrode surface 152 of the electrode member 112. The virtual plane VP preferably lies on the longitudinal axes L1 and L2 in the closed state. That is, in the present embodiment, the virtual plane VP is defined as a plane that is perpendicular to the motion surface T, extends along the longitudinal axes L1 and L2, and passes through the proximity edge 182a of the first electrode surface 152. With respect to the second electrode surface 154 of the electrode member 112, a virtual plane that is perpendicular to the opening and closing directions and passes through the second proximity edge 184a of the second electrode surface 154 in the closed state coincides with the above-described virtual plane VP.

It is assumed that a virtual point exists on the virtual plane VP. The distance between the first electrode surface 152 and the virtual plane VP increases (they separate) as the virtual point moves away from the longitudinal axis L2 toward the first side surface 84a of the treatment portion 26, in a range between the first edge portion 172a of the contact portion 162 or the proximity edge 182a of the first electrode surface 152 and the outer edge 182b of the first electrode surface 152. A distance Ld between the virtual plane VP and the first electrode surface 152 at a position farther from the first proximity edge 182a is larger than a distance Lp between the virtual plane VP and the first electrode surface 152 at a position closer to the first proximity edge 182a in FIG. 3B. The distance between the first outer edge 182b and the virtual plane VP is larger than the distance between the first proximity edge 182a and the virtual plane VP. The distance between the first electrode surface 152 and the virtual plane VP continuously increases from the first proximity edge 182a toward the first outer edge 182b.

A normal vector N2a on the first electrode surface (first surface) 152 is considered. Of the normal vector N2a of the first electrode surface 152, components parallel to the virtual plane VP are directed from the first electrode surface 152 to the first side surface 84a of the treatment portion 26 at any position of the first electrode surface 152. Therefore, of the normal vector N2a of the first electrode surface 152, components parallel to the virtual plane VP are not directed toward the central position M and the second side surface 84b of the treatment portion 26.

In the present embodiment, of the normal vector N1a on the first proximity surface 52a of the facing surface 52 of the blade 44 and the normal vector N2a on the first electrode surface 152 of the blade 102, components parallel to the virtual plane VP are directed toward the first side surface 84a of the treatment portion 26.

The second electrode surface 154 is provided from the second edge portion 172b of the contact portion 162 toward the second side surface 84b of the treatment portion 26.

The second electrode surface 154 includes a second proximity edge 184a that is in proximity to the longitudinal axis L2 and a second outer edge 184b spaced farther from the longitudinal axis L2 than the second proximity edge 184a and in proximity to the second side surface 84b of the treatment portion 26.

It is preferable that there be no clearance between the second edge portion 172b of the contact portion 162 and the proximity edge 184a of the second electrode surface 154, but there may be a slight clearance between the second edge portion 172b and the proximity edge 184a. In addition, it is preferable that there be no step between the second edge portion 172b of the contact portion 162 and the proximity edge 184a of the second electrode surface 154, but there may be a slight step between the second edge portion 172b and the proximity edge 184a. The second electrode surface 154 is provided from the second edge portion 172b of the contact portion 162 or the proximity edge 184a of the second electrode surface 154 toward the first side surface 84a of the treatment portion 26. In the present embodiment, the second electrode surface (second surface) 154 is formed in a flat shape between the second edge portion 172b of the contact portion 162 or the proximity edge 184a of the second electrode surface 154 and the outer edge 184b of the second electrode surface 154. Therefore, in one cross section, the portion between the proximity edge 184a of the second electrode surface 154 and the outer edge 184b of the second electrode surface 154 is linear.

The second electrode surface 154 is inclined in the width direction so as to separate from the side where the first grasping piece 26a is located as the distance to the second side surface 84b of the treatment portion 26 is reduced. In other words, the electrode surface 154 is directed toward the side where the second grasping piece 26b is opened as it separates from the central position M in the width direction.

The distance between the second electrode surface 154 and the virtual plane VP increases (they separate) as the virtual point moves away from the longitudinal axis L2 toward the second side surface 84b of the treatment portion 26 in a range between the second edge portion 172b of the contact portion 162 or the proximity edge 184a of the second electrode surface 154 and the outer edge 184b of the second electrode surface 154. The distance between the second outer edge 184b and the virtual plane VP is larger than the distance between the second proximity edge 184a and the virtual plane VP. The distance between the second electrode surface 154 and the virtual plane VP continuously increases from the second proximity edge 184a to the second outer edge 184b.

A normal vector N2b on the second electrode surface (second surface) 154 is considered. Of the normal vector N2b of the second electrode surface 154, components parallel to the virtual plane VP are directed from the second electrode surface 154 to the second side surface 84b of the treatment portion 26 at any position of the second electrode surface 154. Therefore, of the normal vector N2b of the first electrode surface 154, components parallel to the virtual plane VP are not directed toward the central position M and the first side surface 84a of the treatment portion 26.

In the present embodiment, of the normal vector N1b on the second proximity surface 52c of the facing surface 52 of the blade 44 and the normal vector N2b on the second electrode surface 154 of the blade 102, components parallel to the virtual plane VP are directed toward the second side surface 84b of the treatment portion 26.

In the closed state, the outer edge 182b of the first electrode surface 152 is compared with the first outer edge 56a of the blade 44 that is directed to the first side surface 84a of the treatment portion 26. The outer edge 182b of the first electrode surface 152 is located farther from the longitudinal axes L1 and L2 than the first outer edge 56a of the electrode member 112 of the blade 44. In other words, the outer edge 182b of the first electrode surface (first surface) 152 is separated from the longitudinal axes L1 and L2 more than the first outer edge 56a of the facing surface 52 in the closed state.

Similarly, in the closed state, the outer edge 184b of the second electrode surface 154 is compared with the second outer edge 56b of the blade 44 that is directed to the second side surface 84b of the treatment portion 26. The outer edge 184b of the second electrode surface 154 is located farther from the longitudinal axes L1 and L2 than the second outer edge 56b of the electrode member 112 of the blade 44. In other words, the outer edge 184b of the second electrode surface (second surface) 154 is separated from the longitudinal axes L1 and L2 more than the second outer edge 56b of the facing surface 52 in the closed state.

As the blade 102 of the second grasping piece 26b, the electrode surface 152, the contact portion 162, and the electrode surface 154 as a whole may be formed at an acute angle, a right angle, or an obtuse angle. It is preferable that the width between the first edge portion 172a and the second edge portion 172b of the contact portion 162 be as small as possible. Therefore, the blade 102 of the second grasping piece 26b is preferably formed to be as sharp as possible.

Even when both the central surface 52b and the contact portion 162 are sharp, such a shape is acceptable as long as the state in which both the central surface 52b and the contact portion 162 are in contact with each other can be maintained in the closed state.

(Operation)

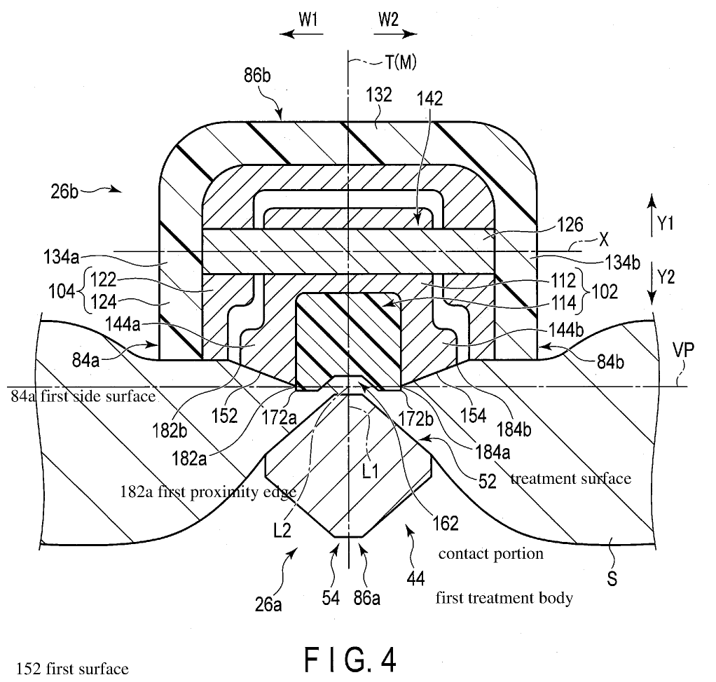

When performing a treatment using the treatment instrument 12, an operator inserts the treatment portion 26 into a body cavity such as an abdominal cavity. Then, a treatment target such as a living tissue (for example, a blood vessel) is disposed between the first grasping piece 26a and the second grasping piece 26b, and the handle 64 is closed with respect to the grip 62. Accordingly, the second grasping piece 26b is closed with respect to the first grasping piece 26a, and the treatment target S is grasped between the first grasping piece 26a and the second grasping piece 26b (see FIG. 4).

An appropriate gripping pressure is applied between the facing surface 52 of the blade 44 of the first grasping piece 26a and the contact portion 162 of the pad member 114 of the blade 102 of the second grasping piece 26b. When the first grasping piece 26a and the second grasping piece 26b are closed, the treatment target S is thinned by the grasping pressure on the motion surface T. Therefore, when the treatment instrument 12 according to the present embodiment is used, an appropriate gripping pressure is applied to the treatment target S between the blade 44 of the first grasping piece 26a and the contact portion 162 of the pad member 114 of the second grasping piece 26b, instead of between electrodes. Further, the treatment target S is brought into contact with the facing surface 52 of the blade 44 of the first grasping piece 26a, and the treatment target S is brought into contact with the first electrode surface 152 and the second electrode surface 154 of the electrode member 112 of the second grasping piece 26b.

In this state, when the treatment target is to be coagulated by a high-frequency current passed through the treatment target, the operator presses the first switch 16a. The system 10 causes the energy source 16 to output electrical energy to the treatment instrument 12 in response to the pressing of the first switch 16a.

The blade 44 of the first grasping piece 26a and the electrode member 112 of the second grasping piece 26b function as electrodes having different potentials with respect to each other. A high-frequency current is passed through the treatment target S grasped between the blade 44 of the first grasping piece 26a and the electrode member 112 of the second grasping piece 26b, and the high-frequency current is applied to the treatment target S as treatment energy. The heat generated in the treatment target S due to the high-frequency current denatures the treatment target S and promotes the coagulation of the treatment target S. That is, the blood vessel or the like, which is the treatment target S, is gelatinized, joined, and sealed by the heat generated in the treatment target S due to the high-frequency current. Thus, the treatment instrument 12 can coagulate or seal (treat) the treatment target.

At this time, in a region between the contact portion 162 and the facing surface (protrusion) 52, the blood vessel which is the treatment target S is dried into a thin paper shape. In addition, the inner peripheral surfaces of the blood vessels are maintained in close contact with each other in the vicinity of the region between the first edge portion 172a of the contact portion 162 and the first proximity surface 52a of the facing surface 52 and in the vicinity of the region between the second edge portion 172b of the contact portion 162 and the second proximity surface 52c of the facing surface 52.

In general, it has been considered that when a treatment instrument for performing treatment using a high-frequency current is used to coagulate a living tissue as a treatment target or to seal a blood vessel as a treatment target in the same manner, opposing electrodes need to have parallel or substantially parallel portions. In other words, it has been considered that when an appropriate treatment is performed with a treatment instrument using a high-frequency current, it is necessary to apply a pressure between electrodes in parallel or substantially parallel to a treatment target.

Here, with respect to the treatment portion 26 of the treatment instrument 12 according to the present embodiment and the treatment portion of the conventional treatment instrument, an experiment of flowing a high-frequency current to the treatment target S and sealing the living tissue was performed under the same conditions. The first treatment body of the treatment portion of the conventional treatment instrument is shaped as a rod configured to allow a high-frequency current to flow and to transmit ultrasonic vibrations, for example, similarly to the blade 44 of the present embodiment described above. In order to simplify the description, the first treatment body of the treatment portion of the conventional treatment instrument has, for example, the same outer shape and material as those of the blade 44 in the present embodiment described above. The second treatment body of the treatment portion of the conventional treatment instrument is configured to allow a high-frequency current to flow, for example, similarly to the blade 102 of the present embodiment described above.

In the treatment portion 26 of the treatment instrument 12 of the present embodiment, the electrode surfaces 152 and 154 of the blade 102 are faced in the directions as described above. Therefore, the region where the pressure is applied to the treatment target S by the second grasping piece 26b is mainly limited to the contact portion 162. In the treatment portion 26 of the treatment instrument 12 according to the present embodiment, since the tissue of the treatment target S escapes in the direction perpendicular to the opening and closing directions, the treatment target S is likely to be thinned by the pressure in the region to which the pressure is applied.

In contrast, the treatment portion of the conventional treatment instrument includes a portion in which the opposing electrodes are parallel or substantially parallel to each other. Therefore, the second treatment body applies pressure to the treatment target not only at the contact portion but also between the electrodes. In the treatment portion of the conventional treatment instrument, the treatment target tends to gather near the contact portion. Therefore, in the treatment portion of the conventional treatment instrument, there is a possibility that the treatment target is less likely to be thinned by the pressure in the region to which the pressure is applied than in the treatment portion 26 of the treatment instrument 12 according to the present embodiment.

The treatment instrument 12 according to the present embodiment and the conventional treatment instrument were subjected to an experiment for sealing the blood vessel of the treatment target S by flowing only the high-frequency current under the same energy output condition. The temperatures of the rod-shaped blade 44 of the treatment portion 26 of the treatment instrument 12 according to the present embodiment and the rod-shaped first treatment body of the treatment portion of the conventional treatment instrument immediately after the experiment and the temperatures of the treated blood vessels were measured.

The temperature of the blade 44 according to the present embodiment immediately after the end of the treatment was lower than the temperature of the conventional first treatment body. On the other hand, the temperature of the treatment target S when treated with the treatment portion 26 of the treatment instrument 12 according to the present embodiment was higher than the temperature of the treatment target when treated with the treatment portion of the conventional treatment instrument.

Then, an experiment was conducted in which blood was allowed to flow through each of the sealed blood vessels. The blood vessel sealing performance was evaluated by measuring a pressure (fluid pressure) at which a fluid such as blood starts to flow through the blood vessel. It was recognized that the blood vessel sealing performance when the treatment instrument 12 according to the present embodiment was used was higher than the blood vessel sealing performance when the conventional treatment instrument was used. As an example, in the experimental values, the value of the blood vessel sealing performance when the treatment instrument 12 according to the present embodiment was used was 1600 mmHg, and the value of the blood vessel sealing performance when the conventional treatment instrument was used was 900 mmHg.

Therefore, by forming the treatment portion 26 of the treatment instrument 12 according to the present embodiment, in particular, the blade 102 of the second grasping piece 26b as described above, it is possible to efficiently increase the temperature of the treatment target S and to suppress the temperature increase of the blade 44 of the first grasping piece 26a. In addition, when the treatment instrument 12 according to the present embodiment is used, since it is possible to suppress the temperature increase of the blade 44 of the first grasping piece 26a, energy is efficiently applied to the treatment target S of the blood vessel. Therefore, in the case of using the treatment instrument 12 according to the present embodiment, the sealing can be performed at a higher speed compared to the case of using the conventional treatment instrument. Further, since the temperature increase of the blade 44 of the first grasping piece 26a can be suppressed, the temperature increase of the non-facing surface 54 (the first back surface 86a of the treatment portion 26) of the blade 44 can be suppressed. Therefore, when the treatment instrument 12 according to the present embodiment is used, invasion of the surrounding tissue due to the non-facing surface 54 of the blade 44 coming into contact with the surrounding tissue during the treatment or immediately after the treatment is suppressed.

Next, an example in which the operator presses the second switch 16b will be described.

When the treatment target is to be incised by transmitting the ultrasonic vibrations to the blade 44 of the first grasping piece 26a, the operator presses the second switch 16b. The system 10 supplies electrical energy from the energy source 16 to the ultrasonic transducer 74 to generate ultrasonic vibrations. The generated ultrasonic vibrations are transmitted from the proximal side to the distal side in the rod 28 and transmitted to the blade 44 of the first grasping piece 26a. At this time, the rod 28 vibrates at any frequency in a predetermined frequency range.

The ultrasonic vibrations transmitted to the blade 44 of the first grasping piece 26a are applied as treatment energy to the grasped treatment target S. At this time, frictional heat is generated between the vibrating first grasping piece 26a and the treatment target S, and the treatment target S is coagulated by the frictional heat and cut at a position between the facing surface (protrusion) 52 of the blade 44 and the contact portion 162 of the blade 102. That is, the blade 102 of the second grasping piece 26b can incise (treat) the treatment target in cooperation with the facing surface (treatment surface) 52 of the blade 44 of the first grasping piece 26a.

As described above, the treatment target S is thinned by the facing surface 52 of the blade 44 of the first grasping piece 26a and the contact portion 162 of the blade 102 of the second grasping piece 26b. At this time, the treatment portion 26 of the treatment instrument 12 according to the present embodiment reduces the volume of the treatment target in the portion to be incised. Accordingly, the cutting speed by the ultrasonic vibrations of the blade 44 is increased and a load is hardly applied to the blade 44; thus, the temperature increase of the blade 44 is suppressed.

Therefore, when the treatment target is incised by transmitting the ultrasonic vibrations to the blade 44 of the treatment instrument 12 according to the present embodiment, the amplitude of the ultrasonic vibration can be reduced even at the same frequency. In addition, when treatment is performed in a liquid, the generation of mist can be suppressed by reducing the amplitude of the longitudinal ultrasonic vibrations along the longitudinal axis L1 of the blades 44, that is, by reducing the vibration speed.

The treatment portion 26 of the treatment instrument 12 according to the present embodiment cuts the treatment target S more easily than the treatment portion of the conventional treatment instrument. Therefore, when ultrasonic vibrations are transmitted to the blade 44 of the treatment instrument 12 in a state in which the treatment target S is appropriately sandwiched by the treatment portion 26, the treatment portion 26 of the treatment instrument 12 according to the present embodiment can incise the treatment target S with less energy than that required by the treatment portion of the conventional treatment instrument. Accordingly, the temperature increase of the blade 44 by the friction between the treatment target and the blade 44 is also suppressed as compared to the conventional first treatment body.

When the second switch 16b is pressed, the system 10 may also cause a high-frequency current to flow through the treatment target together with generation of ultrasonic vibrations. When performing such a treatment, the treatment target is in contact with the electrode surfaces 152 and 154.

As described above, the temperature of the treatment target S efficiently increases while the temperature increase of the blade 44 by the high-frequency current is suppressed. Therefore, drying of the treatment target S easily proceeds, and the incision speed by ultrasonic vibrations of the blade 44 is further increased. At this time, the performance of sealing the treatment target S is favorably maintained.

Therefore, the treatment portion 26 of the treatment instrument 12 according to the present embodiment cuts the treatment target S more easily than the treatment portion of the conventional treatment instrument. Therefore, for example, when the ultrasonic vibrations are transmitted in addition to the high-frequency current, the treatment portion 26 of the treatment instrument 12 according to the present embodiment easily incises the treatment target S with less energy than that required by the treatment portion of the conventional treatment instrument. Accordingly, the temperature increase of the blade 44 by the friction between the treatment target and the blade 44 is also suppressed as compared to the conventional first treatment body.

According to the present embodiment, in the case of performing the incision treatment with energy different from the high-frequency energy, that is, with the ultrasonic vibrations, it is possible to provide the treatment instrument 12 in which the energy can be efficiently applied to the treatment target S by suppressing the temperature increase of the facing surface (treatment surface) 52 of the blade 44 of the first grasping piece 26a and forming the blade 102 of the second grasping piece 26b in an appropriate shape. Therefore, by using the treatment instrument 12 according to the present embodiment, the incision treatment can be efficiently performed on the treatment target with less energy (amount). In addition, in the case of coagulating the treatment target S by using the high-frequency energy, it is possible to provide the treatment instrument 12 in which the temperature increase of the electrode (blade 44) can be suppressed when the high-frequency current is caused to flow through the treatment target S and the energy can be efficiently applied to the treatment target S.

### First Modification

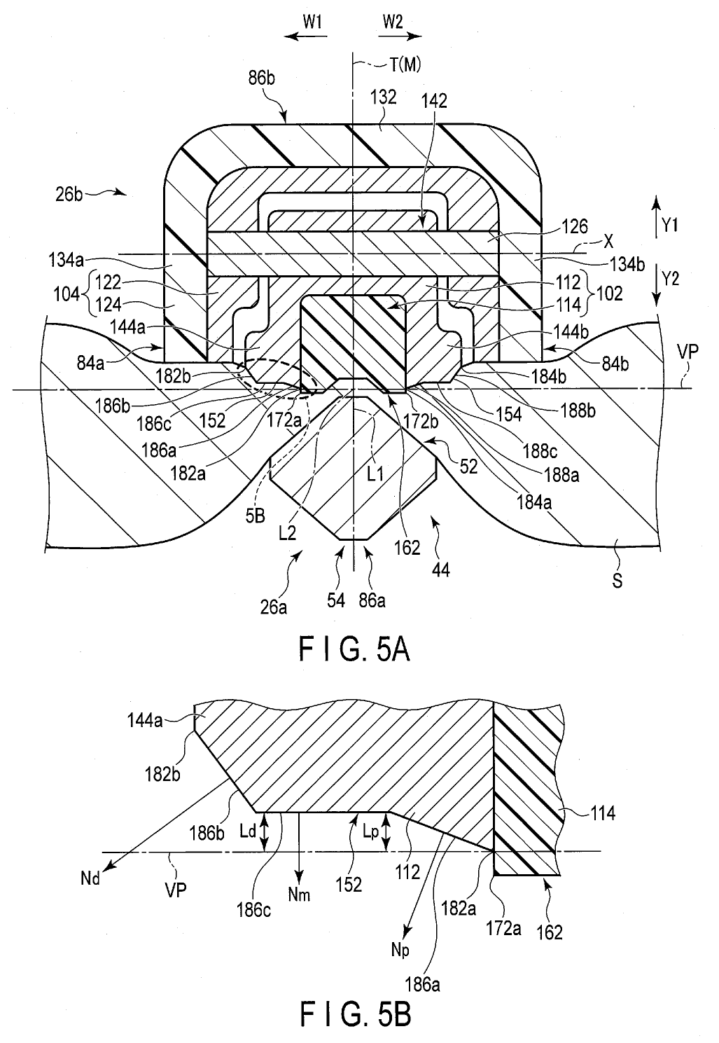

A first modification will be described with reference to FIG. 5A and FIG. 5B. The first modification is a modification of the first embodiment. The same members as those of the first embodiment described above are denoted by the same reference numerals, and detailed descriptions thereof will be omitted. A part of the treatment instrument 12 of the present modification and a part of the treatment instrument 12 of the preceding embodiment may be combined as appropriate. This also applies to the following modifications.

In the first embodiment described above, the entire first electrode surface (first surface) 152 is continuously separated from the virtual plane VP from the longitudinal axis L2 toward the first side surface 84a of the treatment portion 26. Similarly, an example in which the entire second electrode surface (second surface) 154 continues to separate from the virtual plane VP from the longitudinal axis L2 toward the second side surface 84b of the treatment portion 26 has been described.

In the present modification, the first electrode surface (first surface) 152 includes, along the width direction, a first region 186a including a proximity edge 182a, a second region 186b including an outer edge 182b, and a third region 186c formed between the first region 186a and the second region 186b.

The third region 186c is parallel to the virtual plane VP. That is, the region 186c parallel to the virtual plane VP exists in a part of the first electrode surface (first surface) 152. Therefore, in the third region 186c shown in FIG. 5B, the distance Lp between the position close to the first proximity edge 182a and the virtual plane VP is the same as the distance Ld between the position apart from the first proximity edge 182a and the virtual plane VP. In other words, the first electrode surface (first surface) 152 includes the region 186c in which the distance from the virtual plane VP is constant in at least a part of the position between the first edge portion 172a of the contact portion 162 and the outer edge 182b of the first electrode surface (first surface) 152, as the first electrode surface (first surface) 152 separates from the longitudinal axis L2 and approaches the second side surface 84a of the treatment portion 26 in the closed state. Therefore, the distance between the first electrode surface 152 and the virtual plane VP intermittently increases from the first proximity edge 182a toward the first outer edge 182b.

The inclination angles of the first region 186a and the second region 186b with respect to the virtual plane VP may be the same or different.

When the first electrode surface 152 is microscopically viewed, there is a region in which the distance from the virtual plane VP is constant as the distance from the longitudinal axis L2 to the first side surface 84a of the treatment portion 26 is reduced. When macroscopically viewed, the first electrode surface 152 is formed in a state in which the distance from the virtual plane VP is increased as the distance from the longitudinal axis L2 to the first side surface 84a of the treatment portion 26 is reduced. Of the normal vectors Np, Nm, and Nd of the respective regions 186a, 186b, and 186c of the first electrode surface 152, components parallel to the virtual plane VP do not exist (is 0) at any position of the first electrode surface 152 or are directed toward the first side surface 84a of the treatment portion 26. Therefore, of the normal vectors Np, Nm, and Nd of the first electrode surface 152, components parallel to the virtual plane VP are not directed toward the longitudinal axis L2.

Similarly, the second electrode surface (second surface) 154 includes a first region 188a including a proximity edge 184a, a second region 188b including an outer edge 184b, and a third region 188c formed between the first region 188a and the second region 188b. Therefore, the distance between the second electrode surface 154 and the virtual plane VP intermittently increases from the second proximity edge 184a toward the second outer edge 184b.

When the second electrode surface 154 is microscopically viewed, there is a region in which the distance from the virtual plane VP is constant as the distance from the longitudinal axis L2 to the second side surface 84b of the treatment portion 26 is reduced. When macroscopically viewed, the first electrode surface 154 is formed in a state in which the distance from the virtual plane VP is increased as the distance from the longitudinal axis L2 to the second side surface 84b of the treatment portion 26 is reduced. Of the normal vectors N2b of the respective regions 188a, 188b, and 188c of the second electrode surface 154, components parallel to the virtual plane VP do not exist (is 0) at any position of the second electrode surface 154 or are directed toward the second side surface 84b of the treatment portion 26. Therefore, of the normal vector N2b of the second electrode surface 154, components parallel to the virtual plane VP are not directed toward the longitudinal axis L2.

In the case of performing a coagulation treatment on the treatment target using the treatment instrument 12 of this modification, when the first switch 16a is pressed, the coagulation treatment can be performed in the same manner as in the treatment instrument 12 of the first embodiment described above. In addition, in the case of performing an incision treatment on the treatment target using the treatment instrument 12 of this modification, when the second switch 16b is pressed, the incision treatment can be performed in the same manner as in the treatment instrument 12 of the first embodiment described above.

The first electrode surface 152 is formed of the three surfaces 186a, 186c, and 186b from the first proximity edge 182a toward the first outer edge 182b. Therefore, in one cross section, the portion between the proximity edge 182a of the first electrode surface 152 and the outer edge 182b of the first electrode surface 152 is nonlinear as a whole. Similarly, in one cross section, the portion between the proximity edge 184a of the electrode surface 154 and the outer edge 184b of the second electrode surface 154 is nonlinear as a whole.

### Second Modification

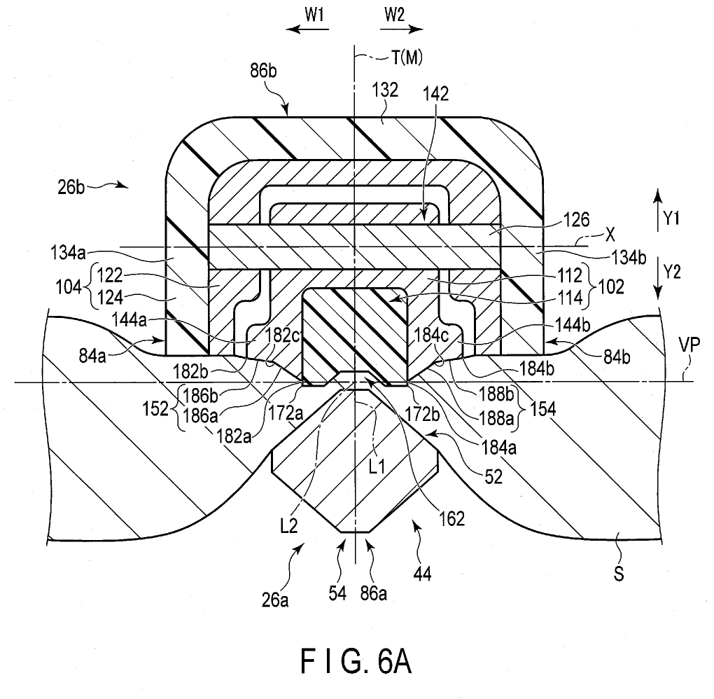

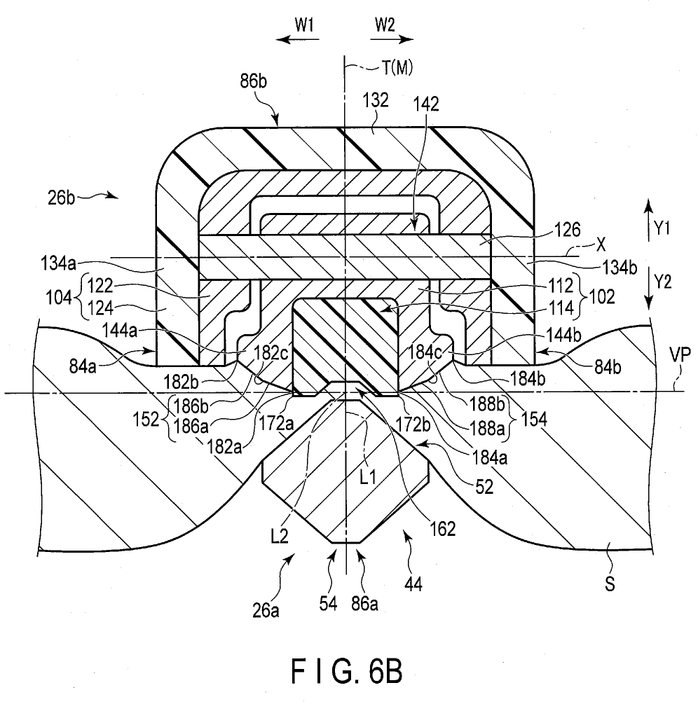

A second modification will be described with reference to FIG. 6A.

In the first modification described above, as an example, the region 186c parallel to the virtual plane VP exists in a part of the first electrode surface (first surface) 152.

As shown in FIG. 6A, in the present modification, the first electrode surface (first surface) 152 includes, along the width direction, the first region 186a including the proximity edge 182a and the second region 186b including the outer edge 182b. That is, the first electrode surface (first surface) 152 includes a plurality of planar regions 186a and 186b.

The planar regions 186a and 186b have different inclination angles with respect to the virtual plane VP. That is, the inclination angles formed by the first region 186a including the first proximity edge 182a and the second region 186b including the first outer edge 182b with respect to the virtual plane VP are different from each other. The angle of a boundary position 182c between the planar regions 186a and 186b is an obtuse angle larger than 90° and smaller than 180°. The first electrode surface 152 is formed in a state in which the distance from the virtual plane VP is increased as the distance from the longitudinal axis L2 to the first side surface 84a of the treatment portion 26 is reduced. Therefore, the distance between the first electrode surface 152 and the virtual plane VP continuously increases from the first proximity edge 182a toward the first outer edge 182b.

Similarly, the second electrode surface (second surface) 154 includes, along the width direction, the first region 188a including the proximity edge 184a and the second region 188b including the outer edge 184b. That is, the second electrode surface (second surface) 154 includes a plurality of planar regions 188a and 188b.

The planar regions 188a and 188b have different inclination angles with respect to the virtual plane VP. That is, the inclination angles formed by the first region 188a including the second proximity edge 184a and the second region 188b including the second outer edge 184b with respect to the virtual plane VP are different from each other. The angle of a boundary position 184c between the planar regions 188a and 188b is an obtuse angle larger than 90° and smaller than 180°. The second electrode surface 154 is formed in a state in which the distance from the virtual plane VP is increased as the distance from the longitudinal axis L2 to the second side surface 84b of the treatment portion 26 is reduced. Therefore, the distance between the second electrode surface 154 and the virtual plane VP continuously increases from the second proximity edge 184a toward the second outer edge 184b.

Even when the first electrode surface 152 and the second electrode surface 154 are formed in this manner, an appropriate treatment can be performed in the same manner as in the treatment instrument 12 of the first embodiment described above.

Here, the example in which the first electrode surface 152 includes the two regions 186a and 186b has been described. In the case where the first electrode surface 152 includes three planar regions, another boundary is formed on the first electrode surface 152 separately from the position indicated by the reference sign 182c. Thus, two boundaries (two angles) are formed on the first electrode surface 152. In any case, the first electrode surface 152 is formed in a state in which the distance from the virtual plane VP is increased as the distance from the longitudinal axis L2 to the first side surface 84a of the treatment portion 26 is reduced. The same applies to the case where two boundaries (two angles) are formed on the second electrode surface 154.

### Third Modification

A third modification will be described with reference to FIG. 6B. This modification is a further modification of the second modification.

As shown in FIG. 6B, in the present modification, the first electrode surface (first surface) 152 includes, along the width direction, a first region 186a including a proximity edge 182a and a second region 186b including an outer edge 182b. That is, the first electrode surface (first surface) 152 includes a plurality of planar regions 186a and 186b.

The planar regions 186a and 186b have different inclination angles with respect to the virtual plane VP. The angle of a boundary position 182c between the planar regions 186a and 186b is a reflex angle larger than 180° and smaller than 270°. That is, the first electrode surface 152 of the present modification is different from the first electrode surface 152 of the second modification in the angle of the boundary position 182c. The first electrode surface 152 is formed in a state in which the distance from the virtual plane VP is increased as the distance from the longitudinal axis L2 to the first side surface 84a of the treatment portion 26 is reduced. Therefore, the distance between the first electrode surface 152 and the virtual plane VP continuously increases from the first proximity edge 182a toward the first outer edge 182b.

Similarly, the second electrode surface (second surface) 154 includes, along the width direction, the first region 188a including the proximity edge 184a and the second region 188b including the outer edge 184b. That is, the second electrode surface (second surface) 154 includes a plurality of planar regions 188a and 188b.

The planar regions 188a and 188b have different inclination angles with respect to the virtual plane VP. The angle of a boundary position 184c between the planar regions 188a and 188b is a reflex angle larger than 180° and smaller than 270°. That is, the second electrode surface 154 of the present modification is different from the second electrode surface 154 of the second modification in the angle of the boundary position 182c. The second electrode surface 154 is formed in a state in which the distance from the virtual plane VP is increased as the distance from the longitudinal axis L2 to the second side surface 84b of the treatment portion 26 is reduced. Therefore, the distance between the second electrode surface 154 and the virtual plane VP continuously increases from the second proximity edge 184a toward the second outer edge 184b.

Even when the first electrode surface 152 and the second electrode surface 154 are formed in this manner, an appropriate treatment can be performed in the same manner as in the treatment instrument 12 of the first embodiment described above.

### Fourth Modification

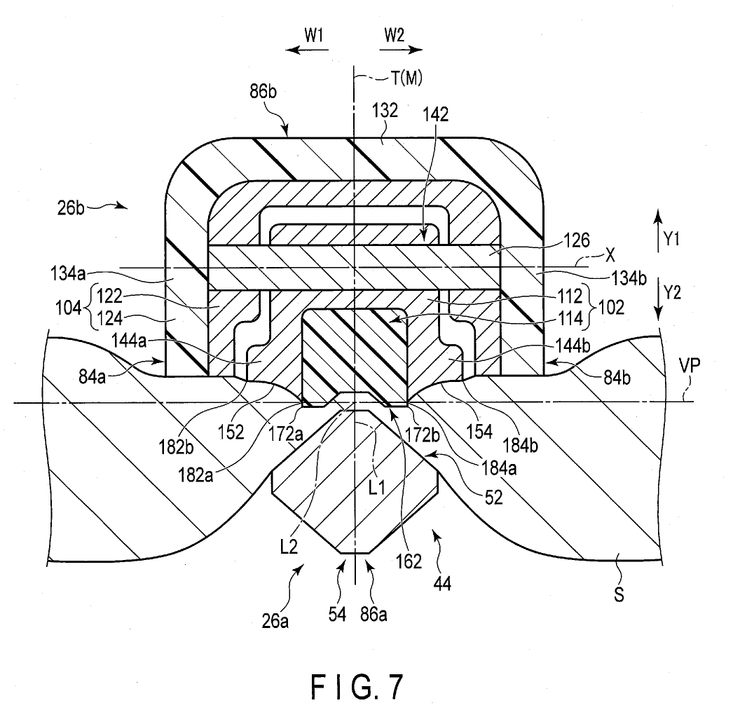

A fourth modification will be described with reference to FIG. 7.

In the first embodiment described above, the entire first electrode surface (first surface) 152 separates from the virtual plane VP in a planar manner as the distance from the longitudinal axis L2 to the first side surface 84a of the treatment portion 26 is reduced. Similarly, the entire second electrode surface (second surface) 154 separates from the virtual plane VP in a planar manner as the distance from the longitudinal axis L2 to the second side surface 84b of the treatment portion 26 is reduced.

In the present modification, the entire first electrode surface (first surface) 152 separates from the virtual plane VP in a curved surface shape (nonlinear in cross section) as the distance from the longitudinal axis L2 to the first side surface 84a of the treatment portion 26 is reduced. Thus, in the present embodiment, the first electrode surface (first surface) 152 is formed in a curved surface shape between the first edge portion 172a of the contact portion 162 or the proximity edge 182a of the first electrode surface 152 and the outer edge 182b of the first electrode surface 152. Therefore, in one cross section, the portion between the proximity edge 182a of the first electrode surface 152 and the outer edge 182b of the first electrode surface 152 is nonlinear.

A normal vector N2a on the first electrode surface (first surface) 152 is considered (see FIG. 2 and FIG. 3A). Of the normal vector N2a of the first electrode surface 152, components parallel to the virtual plane VP are directed to the first side surface 84a of the treatment portion 26 at any position of the first electrode surface 152.

The second electrode surface (second surface) 154 is formed symmetrically with respect to the first electrode surface (first surface) 152 with respect to the motion surface T (central position M) in the width direction. A normal vector N2b on the second electrode surface (second surface) 154 is considered (see FIG. 2 and FIG. 3A). Of the normal vector N2b of the second electrode surface 154, components parallel to the virtual plane VP are directed to the second side surface 84b of the treatment portion 26 at any position of the second electrode surface 154.

Even in this case, as in the first embodiment and the first modification described above, when treatment is performed, it is possible to exhibit better treatment performance than the conventional treatment instrument.

The example in which the first electrode surface 152 and the second electrode surface 154 are concave curved surfaces has been described. Although not shown, the first electrode surface 152 and the second electrode surface 154 may be convex curved surfaces.

Although not shown, the first electrode surface 152 may be a combination of a portion that separates from the virtual plane VP in a planar shape and a portion that separates from the virtual plane VP in a curved shape as the distance from the longitudinal axis L2 to the first side surface 84a of the treatment portion 26 is reduced. That is, the first electrode surface 152 is preferably formed of one or more flat surfaces and one or more curved surfaces.

Similarly, the second electrode surface 154 may be a combination of a portion that separates from the virtual plane VP in a planar shape and a portion that separates from the virtual plane VP in a curved shape as the distance from the longitudinal axis L2 to the second side surface 84b of the treatment portion 26 is reduced. That is, the second electrode surface 154 is preferably formed of one or more flat surfaces and one or more curved surfaces.

### Fifth Modification

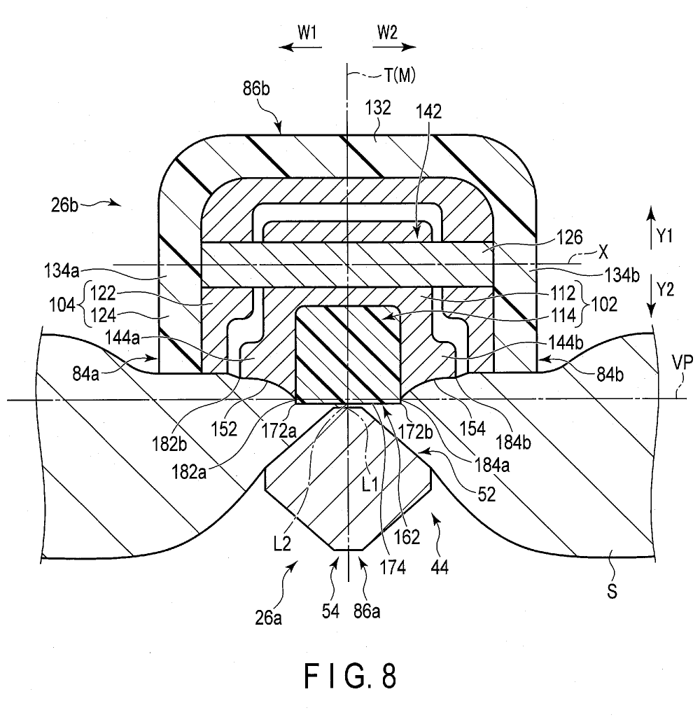

A fifth modification will be described with reference to FIG. 8.

As illustrated in FIG. 8, the contact portion 162 of the pad member 114 includes a first edge portion 172a separated from the central position M in the first width direction W1, a second edge portion 172b separated from the central position M in the second width direction W2, and a central portion 174 through which the central position M passes, as in the first embodiment described above.

The central portion 174 is formed between the first edge portion 172a and the second edge portion 172b. In the present modification, the central portion 174 is not recessed, but is formed in a flat shape flush with the first edge portion 172a and the second edge portion 172b. Therefore, in the present modification, the central surface 52b of the facing surface 52 can be brought into contact with the central portion 174 of the contact portion 162.

Although the first electrode surface 152 and the second electrode surface 154 in FIG. 8 are shown in the same shape as the example shown in FIG. 7, it is needless to say that they may have the shapes shown in FIG. 2 to FIG. 4, FIG. 5A, FIG. 5B, FIG. 6A, and FIG. 6B.

### Sixth Modification

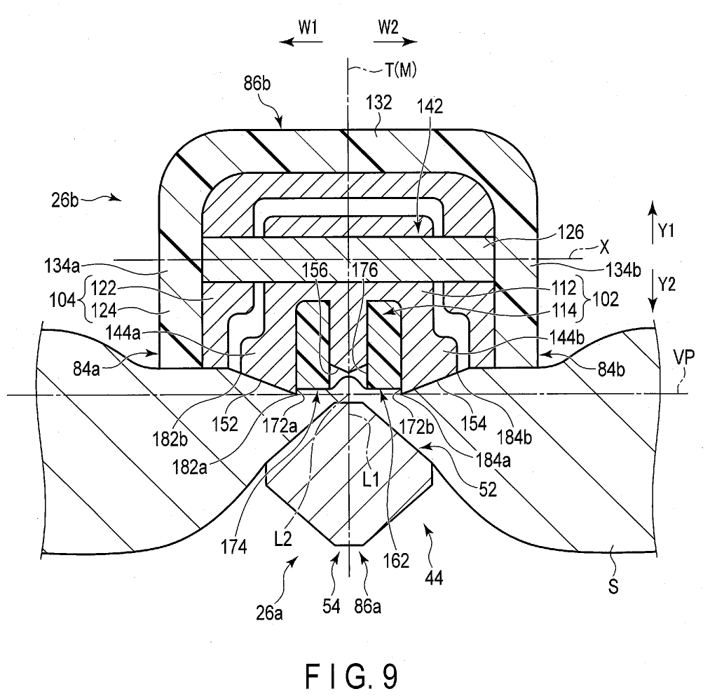

A sixth modification will be described with reference to FIG. 9.

In the examples described above, the first edge portion 172a of the contact portion 162 of the first grasping piece 26b is farther from the second back surface 86b of the treatment portion 26 than the proximity edge 182a of the first electrode surface 152 of the electrode member 112 along the opening and closing directions.

As shown in FIG. 9, the first edge portion 172a of the contact portion 162 may be located at an equal distance to the second back surface 86b of the treatment portion 26 from the proximity edge 182a of the second electrode surface 152 of the electrode member 112 along the opening and closing directions or may be located closer to the second back surface 86b than the proximity edge 482a. Similarly, the second edge portion 172b of the contact portion 162 may be located at an equal distance to the second back surface 86b of the treatment portion 26 from the proximity edge 184a of the second electrode surface 154 of the electrode member 112 along the opening and closing directions or may be located closer to the second back surface 86b than the proximity edge 184a. Therefore, in the present modification, the virtual plane VP passing through the proximity edge 182a of the electrode surface 152 and the proximity edge 184a of the electrode surface 154 may not intersect the pad member 114 including the contact portion 162.

A recess (concave portion) 176 is formed in the central portion 174 of the pad member 114 of the second grasping piece 26b shown in FIG. 9. That is, the contact portion 162 includes the recess 176 recessed in a direction opposite to a direction toward the facing surface 52 of the blade 44 of the first grasping piece 26a between the first edge portion 172a and the second edge portion 172b.

The blade 102 of the second grasping piece 26b includes, in the recess 176, an electrode surface (third surface) 156 to be used as a high-frequency electrode having the same potential as that of the first electrode surface 152 and the second electrode surface 154.

In a state in which the treatment target S is gripped between the first grasping piece 26a and the second grasping piece 26b, the treatment target S may come into contact with the electrode surface 156 in the recess 176 before the high-frequency current is applied or while the high-frequency current is being applied. When the treatment target S comes into contact with the electrode surface 156, a current flows not only between the first electrode surface 152 and the facing surface 52 and between the second electrode surface 154 and the facing surface 52, but also between the electrode surface 156 and the facing surface 52.

Even in this case, when treatment is performed, it is possible to exhibit better treatment performance than the conventional treatment instrument, as in the first embodiment and the first to fifth modifications described above.

The positional relationship between the first proximity edge 182a of the first electrode surface 152 and the second proximity edge 184a of the second electrode surface 154, and the first edge portion 172a and the second edge portion 172b of the contact portion 162, can also be applied to the treatment instrument 12 described in the first to fifth modifications.

The electrode surface 156 can be similarly used for the blade 102 of the first embodiment and the first to fifth modifications described above.

### Second Embodiment

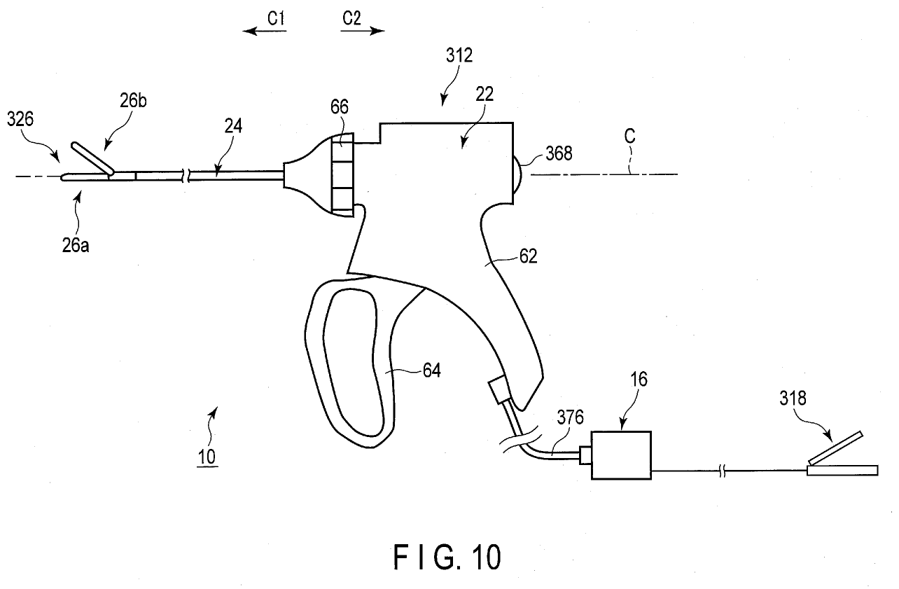

A second embodiment will be described with reference to FIG. 10 to FIG. 15. The second embodiment is a modification of the first embodiment including its modifications. The same members as those of the first embodiment described above are denoted by the same reference numerals, and detailed descriptions thereof will be omitted. A part of a treatment instrument 312 of the present modification and a part of the treatment instrument 12 of the preceding embodiment may be combined as appropriate. This also applies to the following modifications.

In the following, a treatment instrument 312 that performs a treatment on a living tissue using heat and a high-frequency current will be described. In the present embodiment, along with the flow of the high-frequency energy or separately from the flow of the high-frequency energy, heat from a heat generating portion (heater) 344c is input to a blade (first treatment body) 344 of a treatment portion 326 to be described later as another energy (second energy) different from the high-frequency energy.

A treatment system 10 comprises the treatment instrument 312 and an energy source 16.

The treatment instrument 312 is removably connected to the energy source 16 via a cable 376. The treatment instrument 312 includes a housing 22, a tubular shaft (sheath) 24, and the treatment portion (end effector) 326. In the present embodiment, the longitudinal axis C is defined as a straight central axis with respect to a relay portions 328 (to be described later) of the treatment portion 326.

An operating device 318 such as a foot switch is electrically connected to the energy source 16. The operating device 318 preferably includes a plurality of pedals. In the operating device 318, an operation for outputting electrical energy from the energy source 16 to the treatment instrument 312 is input. In one embodiment, instead of the operating device 318 separate from the treatment instrument 312, or in addition to the operating device 318 separate from the treatment instrument 312, an operation switch (not shown) or the like attached to the housing 22 or the like of the treatment instrument 312 is provided as the operating device. Then, an operation for outputting electrical energy from the energy source 16 to the treatment instrument 312 is input by an operating device attached to the treatment instrument 312.

In the present embodiment, one end of the cable 376 is connected to the grip 62. The other end of the cable 376 is removably connected to the energy source 16.

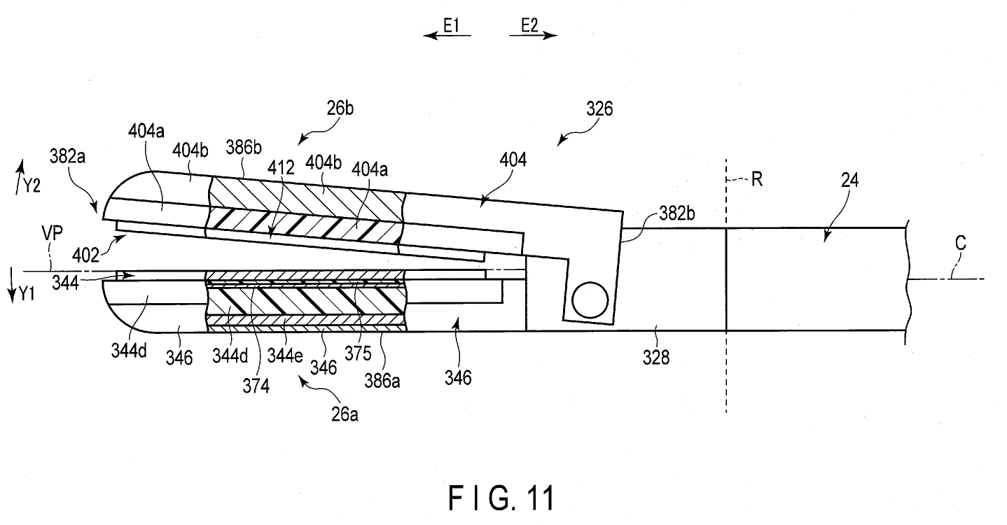

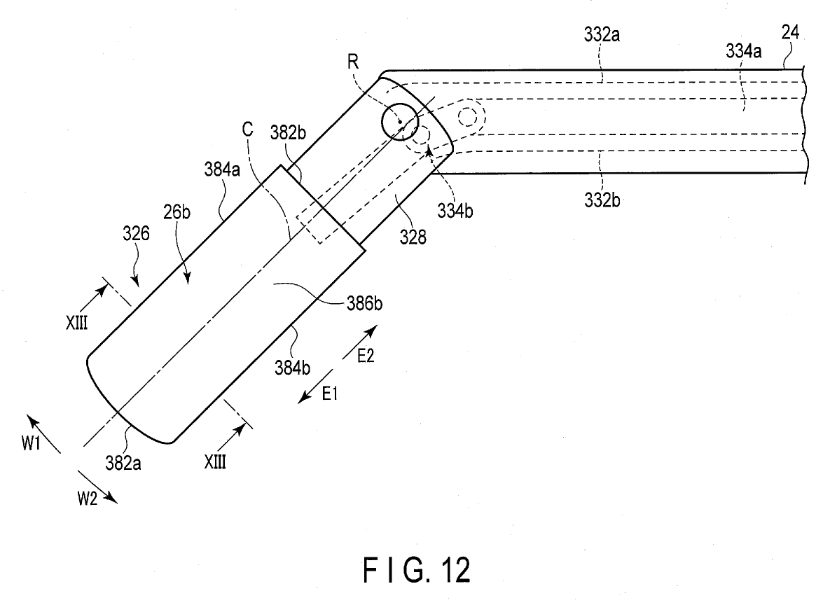

FIG. 11 and FIG. 12 are views showing an example of the configuration of the distal end portion of the shaft 24 and the treatment portion 326. As shown in FIG. 11 and FIG. 12, the treatment portion 326 includes a proximal end and a distal end, and extends from the proximal end to the distal end along the longitudinal direction (the direction indicated by the arrow E1 and the arrow E2). The treatment portion 326 is coupled to the distal end portion of the shaft 24. The treatment portion 326 is rotatable with respect to the shaft 24 about the coupling position with the shaft 24, that is, rotatable about a rotation axis R. Rotation of the treatment portion 326 about the rotation axis R relative to the shaft 24 causes the treatment portion 326 to bend relative to the shaft 24 and the longitudinal axis C. In a state where the treatment portion 326 is not bent with respect to the shaft 24, the longitudinal direction of the treatment portion 326 is parallel or substantially parallel to the axial direction of the shaft 24 and is parallel or substantially parallel to the longitudinal axis C.

Here, the rotation axis R extends along a direction intersecting (perpendicular or substantially perpendicular to) the longitudinal direction of the treatment portion 326. The bending direction of the treatment portion 326 (the same direction as the width direction indicated by the arrow W1 and the arrow W2) intersects (is perpendicular or substantially perpendicular to) the longitudinal direction of the treatment portion 326 and intersects (is perpendicular or substantially perpendicular to) the rotation axis R. In the present embodiment, the bending direction of the treatment portion 326 is parallel or substantially parallel to the width direction of the treatment portion 326. FIG. 11 shows a state viewed from one side in the width direction of the treatment portion 326, and a part thereof is shown in a cross section perpendicular or substantially perpendicular to the width direction of the treatment portion 326. FIG. 12 shows a state viewed from one side in a direction parallel or substantially parallel to the rotation axis R of the treatment portion 326, and also shows an internal structure of the shaft 24.

In the present embodiment, an operation dial 368 is attached to the housing 22 as an operation member. A pair of elongated members 332a and 332b extend along the longitudinal axis C, namely, along the axial direction of the shaft 24. The distal end of each of the elongated members 332a and 332b is connected to the treatment portion 326. When an operation is input to the operation dial 368, a driving force is transmitted to the elongated members 332a and 332b via a driving force transmission mechanism (not shown) or the like inside the housing 22, and each of the elongated members 332a and 332b moves along the longitudinal axis C with respect to the shaft 24. Accordingly, the treatment portion 326 rotates about the rotation axis R, and the treatment portion 326 performs a bending operation with respect to the shaft 24.

The treatment portion 326 includes a tubular relay portion 328 and a pair of grasping pieces 26a and 26b. The relay portion 328 is attached to the distal end portion of the shaft 24 so as to be rotatable about the rotation axis R. The pair of grasping pieces 26a and 26b are openable and closable relative to each other. One of the grasping pieces 26a and 26b is rotatably attached to the relay portion 328. In one embodiment, the other of the grasping pieces 26a and 26b is integrally formed with the relay portion 328 or is fixed to the relay portion 328. In another embodiment, the other of the grasping pieces 26a is 26b is also rotatably attached to the relay portion 328. In another embodiment, a rod member (not shown) is provided that protrudes distally from the distal end of the relay portion 328. A portion of the rod member protruding from the relay portion 328 forms the other of the grasping pieces 26a and 26b.

In the present embodiment, the opening and closing directions of the grasping pieces 26a and 26b (the directions indicated by the arrow Y1 and the arrow Y2), that is, the moving directions of the grasping pieces 26a and 26b in the opening operation and the closing operation of the treatment portion 326 intersect (are perpendicular or substantially perpendicular to) the longitudinal direction of the treatment portion 326 and intersect (are perpendicular or substantially perpendicular to) the bending direction of the treatment portion 326. The opening and closing directions of the grasping pieces 26a and 26b (the opening and closing directions of the treatment portion 326) are parallel or substantially parallel to the rotation axis R.

A movable member 334a extends along the axial direction of the shaft 24 inside or outside the shaft 24. The proximal end portion of the movable member 334a is coupled to the handle 64 inside the housing 22. The distal end of the movable member 334a is connected to the treatment portion 326 via a link mechanism 334b. Thus, the link mechanism 334b connects the treatment portion 326 and the movable member 334a. Since the link mechanism 334b is provided, when the treatment portion 326 is bent with respect to the shaft 24, the treatment portion 326 is also bent with respect to the movable member 334a.

When the handle 64 is opened or closed with respect to the grip 62, the movable member 334a moves along the axial direction of the shaft 24. As a result, the driving force from the movable member 334a is transmitted to the treatment portion 326 through the link mechanism 334b, and the grasping pieces 26a and 26b in pair are opened or closed with respect to each other. The closing of the grasping pieces 26a and 26b relative to each other allows the grasping pieces 26a and 26b to clamp tissue or the like between the grasping pieces 26a and 26b.

In whichever position the movable member 334a is located in the axial direction of the shaft 24, the shaft 24 is coupled to the treatment portion 326 in a range in which the link mechanism 334b extends. In other words, in whichever position the movable member 334a is located in the direction along the axial direction of the shaft 24, the coupling position of the treatment portion 326 to the shaft 24 is located in a range in which the link mechanism 334b extends.

In one embodiment, the treatment portion 326 is non-bendable relative to the shaft 24. In this case, the link mechanism 334b is not provided, and the distal end of the shaft 24 is directly connected to the treatment portion 326. At this time, the longitudinal axis C of the relay portion 328 coincides with the longitudinal axis of the shaft 24.

In this case, the operation dial 368 and the elongated members 332a and 332b are not provided, and the longitudinal direction of the treatment portion 326 is always parallel or substantially parallel to the axial direction of the shaft 24. In this embodiment, one of the grasping pieces 26a and 26b is rotatably attached to the distal end portion of the shaft 24. The other of the grasping pieces 26a and 26b may be formed integrally with the shaft 24 or may be fixed to the shaft 24. The other of the grasping pieces 26a and 26b may also be rotatably attached to the shaft 24.

In the treatment portion 326 of the present embodiment, the dimension of the treatment portion 326 in the longitudinal direction is larger than each of the dimension of the treatment portion 326 in the opening and closing directions and the dimension of the treatment portion 326 in the width direction. In the longitudinal direction of the treatment portion 326, the respective dimensions of the first grasping piece 26a and the second grasping piece 26b are the same or substantially the same. In the width direction of the treatment portion 326, the respective dimensions of the first grasping piece 26a and the second grasping piece 26b are the same or substantially the same, unlike the treatment portion 26 of the first embodiment.

The treatment portion 326, in which the first grasping piece 26a and the second grasping piece 26b cooperate, includes a distal end portion 382a, a proximal end portion 382b, a first side surface (side portion) 384a, a second side surface (side portion) 384b, a first back surface 386a, and a second back surface 386b. In particular, the first back surface 386a is formed on the first grasping piece 26a and the second back surface 386b is formed on the second grasping piece 26b.

In the present embodiment, the widths of the first grasping piece 26a and the second grasping piece 26b are substantially the same. Therefore, the first side surface 384a is defined by a virtual line connecting a position of a jaw 346 of the first grasping piece 26a and/or a base 344d of the blade 344 farthest from a motion surface T in the first width direction W1 and a position of a jaw 404 of the second grasping piece 26b and/or a base 414 of a blade 402 farthest from the motion surface T in the first width direction W1. Similarly, the second side surface 384b is defined by a virtual line connecting a position of the jaw 346 of the first grasping piece 26a and/or the base 344d of the blade 344 farthest from the motion surface T in the second width direction W2 and a position of the jaw 404 of the second grasping piece 26b and/or the base 414 of the blade 402 farthest from the motion surface T in the second width direction W2.

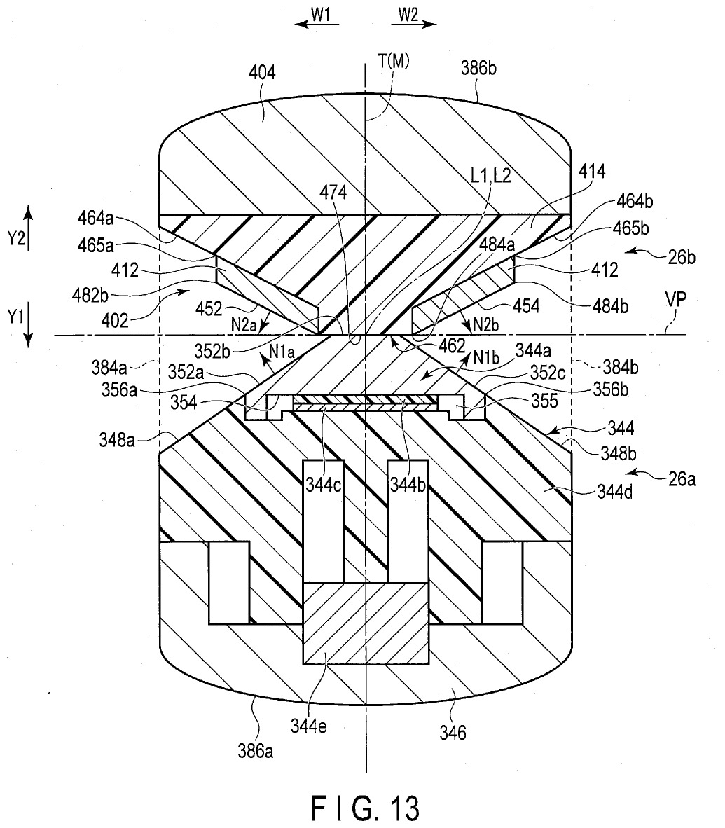

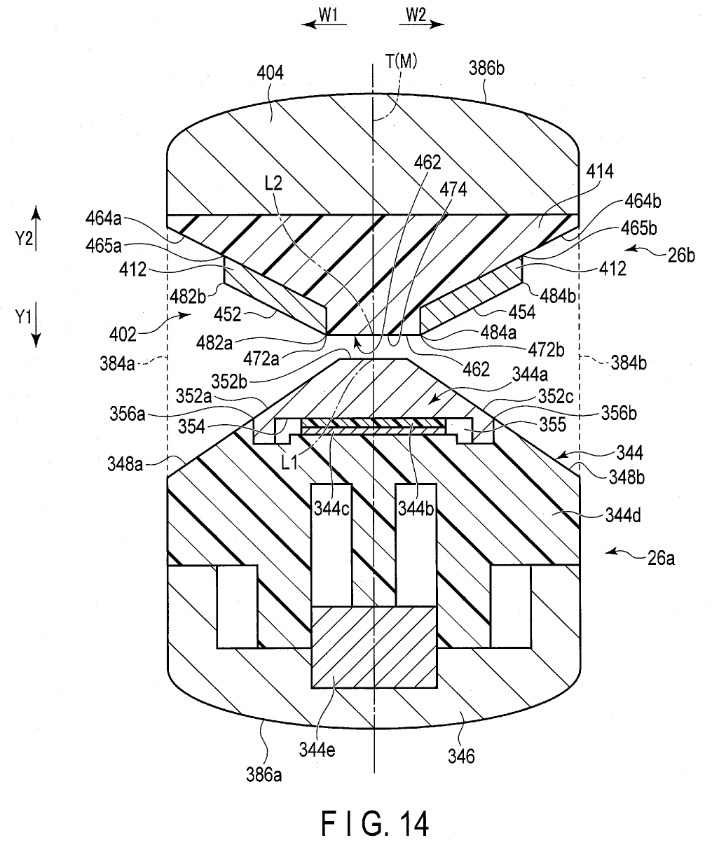

FIG. 13 and FIG. 14 show cross sections perpendicular or substantially perpendicular to the longitudinal direction of the treatment portion 326. The first grasping piece 26a and the second grasping piece 26b of the treatment portion 326 are openable and closable relative to each other.

The first grasping piece 26a includes the blade (first treatment body) 344 and the jaw (support body) 346 provided with the blade 344. The blade 344 is attached to the jaw 346 from the side where the second grasping piece 26b is located. In the first grasping piece 26a, the blade 344 is openable and closable, together with the jaw 346, relative to the second grasping piece 26b.

The second grasping piece 26b includes the blade (second treatment body) 402 and the jaw (support body) 404 provided with the blade 402. The blade 402 is attached to the jaw 404 from the side where the first grasping piece 26a is located. In the second grasping piece 26b, the blade 402 is openable and closable, together with the jaw 404, relative to the first grasping piece 26a.

The opening and closing directions of the treatment portion 326 relatively approaching and separating from each other are defined by rotating the second grasping piece 26b with respect to the first grasping piece 26a. The opening and closing directions intersect the extending direction of the treatment portion 326 with respect to the distal end of the relay portion 328 along the longitudinal axis C, for example, substantially perpendicularly.

As described above regarding the first embodiment, in the state in which the second grasping piece 26b is opened with respect to the first grasping piece 26a (see FIG. 14), the longitudinal axes L1 and L2 deviate from each other. The longitudinal axis L2 moves with respect to the longitudinal axis C as the second grasping piece 26b rotates with respect to the relay portion 328. In the state in which the second grasping piece 26b is closed with respect to the first grasping piece 26a shown in FIG. 13, the longitudinal axes L1 and L2 coincide.

The blade (first treatment body) 344 includes an electrode member 344a, an adhesive layer 344b provided on the back surface of the electrode member 344a and having an electrical insulating property, a heat generating portion 344c provided on the back surface of the adhesive layer 344b, a base 344d provided between the electrode member 344a and the heat generating portion 344c on one hand and the jaw (first jaw) 346 on the other, and a heat sink 344e provided between the base 344d and the jaw 346.

The electrode member 344a continuously extends over a range from the proximal end portion to the distal end portion of the first grasping piece 26a in the longitudinal direction of the treatment portion 326. The electrode member 344a has electrical conductivity and high heat conductivity. The electrode member 344a is formed of, for example, an aluminum alloy or a metal that contains aluminum.

Each of the base 344d, the heat sink 344e, and the jaw 346 continuously extends over a range from the proximal end portion to the distal end portion of the first grasping piece 26a in the longitudinal direction of the treatment portion 326. In the blade 344, the heat sink 344e is attached to the base 344d from the side where the jaw 346 opens (from the back side of the jaw 346). The jaw 346 is attached to the base 344d and the heat sink 344e from the side where the jaw 346 opens. The jaw 346 forms the back surface 386a facing the opening side of the jaw 346 on the outer surface of the jaw 346. The electrode member 344a is attached to the base 344d from the side where the jaw 346 is closed, that is, the side where the blade 402 is located.

The base 344d is electrical insulating and has a lower heat conductivity than the electrode member 344a. The base 344d is made of resin such as liquid crystal polymer (LCP) and polyetheretherketone (PEEK). The heat sink 344e has a higher heat conductivity than the base 344d, and transfers the heat transferred through the base 344d to the proximal side of the jaw 346. The heat sink 344e is made of a material having high heat conductivity, such as aluminum or copper. The jaw 346 is made of metal. The exposed portion of the jaw 346, including the first back surface 386a of the first grasping piece 26a, is preferably coated with an electrically insulative coating or overmolded with an electrically insulative material.

The electrode member 344a includes a protrusion (facing surface) 352 protruding towards the blade 402 of the second grasping piece 26b. The protrusion 352 protrudes toward the side Y2 where the jaw (first jaw) 346 is closed. In addition, the protrusion 352 continuously extends over a range from the proximal end portion to the distal end portion of the jaw 346 in the longitudinal direction of the treatment portion 326. The electrode member 344a includes an electrode back surface 354 facing the side Y1 opposite to the side Y2 from which the protrusion 352 protrudes. The electrode back surface 354 faces the side where the jaw 346 opens, and is not exposed to the outside. The base 344d is attached to the electrode back surface 354 of the electrode member 344a, and a cavity 355 is defined between the base 344d and the electrode back surface 354 of the electrode member 344a. Each of the electrode back surface 354 and the cavity 355 continuously extends over the range from the proximal end portion to the distal end portion of the jaw 346 in the longitudinal direction of the treatment portion 326.

The heat generating portion 344c such as a heater is arranged in the cavity 355. The heat generating portion 344c includes a heater wire (not shown), and the heater wire is formed of an electrical conductive material such as stainless steel, platinum, and tungsten. The heat generating portion 344c is attached to the electrode back surface 354 of the electrode member 344a via the adhesive layer 344b. The heat generating portion 344c is electrically insulated from the electrode member 344a by the adhesive layer 344b. Each of the heat generating portion 344c and the adhesive layer 344b extends continuously over the range from the proximal end portion to the distal end portion of the jaw 346 in the longitudinal direction of the treatment portion 326.

The first grasping piece 26a is symmetrical or substantially symmetrical in the width direction about the motion surface T (central position M). In the present embodiment, the motion surface T passes through the protrusion 352 and the heat generating portion 344c. The electrode member 344a is also symmetrical or substantially symmetrical about the central position M.

The protrusion 352 may be of a flat surface or a curved surface. The protrusion 352 may be a combination of a plurality of curved surfaces and/or flat surfaces. In the present embodiment, the protrusion 352 includes three surfaces 352a, 352b, and 352c. The surface (first proximity surface) 352a is in proximity to a first side surface 384a (described later) of the treatment portion 326. The surface 352b is formed as a central surface in a central portion in the width direction of the protrusion 352. The surface (second proximity surface) 352c is proximal to a second side surface 384b (described later) of the treatment portion 326.

The base 344d includes a pair of facing surfaces (insulating surfaces) 348a and 348b spaced apart from each other. In FIG. 13 to FIG. 15, the facing surface 348a and the first proximity surface 352a are formed to be flush with each other, and the facing surface 348b and the second proximity surface 352c are formed to be flush with each other.

The electrode member 344a of the blade 344 of the first grasping piece 26a includes a first outer edge 356a and a second outer edge 356b. In the present embodiment, the first outer edge 356a is a boundary between the first proximity surface 352a of the protrusion 352 and the facing surface 348a of the base 344d. The second outer edge 356b of the electrode member 344a of the blade 344 of the first grasping piece 26a is a boundary between the surface 352c of the protrusion 352 and the facing surface 348b of the base 344d.

The first outer edge 356a is closer to the first side surface 384a of the treatment portion 326 than to the second side surface 384b of the treatment portion 326. Therefore, the first outer edge 356a of the blade 344 is brought in proximity to the first side surface 384a of the treatment portion 326 separated from the longitudinal axis L1 of the blade 344 in the first width direction W1 perpendicular to the opening and closing directions along the motion surface T.

The second outer edge 356b is closer to the second side surface 384b of the treatment portion 326 than to the first side surface 384a of the treatment portion 326. Therefore, the second outer edge 356b of the blade 344 is brought in proximity to the second side surface 384b of the treatment portion 326 separated from the longitudinal axis L1 of the blade 344 in the second width direction W2 perpendicular to the opening and closing directions along the motion surface T.

In the present embodiment, the central surface 352b of the protrusion 352 between the first outer edge 356a and the second outer edge 356b of the blade 344 is used as a protrusion that is brought into contact with a contact portion (contact surface) 462 (to be described later) of the blade 402 of the second grasping piece 26b in cooperation with a region of the surfaces 352a and 352c that are adjacent to the central surface 352b.

The blade 402 includes an electrode member 412 and the base 414.

Each of the base 414 and the jaw (second jaw) 404 continuously extends over the range from the proximal end portion to the distal end portion of the jaw 404 in the longitudinal direction of the treatment portion 326. The jaw 404 is attached to the base 414 from the side where the jaw 404 opens. The jaw 404 forms the second back surface 386b facing the side where the jaw 404 opens on the outer surface of the jaw 404. In addition, for example, the electrode member 412 is attached to the base 414 from the side where the jaw 404 is closed, that is, the side where the blade 344 is located. The electrode member 412 continuously extends over the range from the proximal end portion to the distal end portion of the second grasping piece 26b in the longitudinal direction of the treatment portion 326. The electrode member 412 is formed of an electrical conductive material.

The base 414 is electrical insulating and has a lower heat conductivity than the electrode member 412. The base 414 is made of resin, for example. The jaw 404 is made of metal. The exposed portion of the jaw 404, including the second back surface 386b, is preferably coated with an electrically insulative coating or overmolded with an electrically insulative material.

The base (second base) 414 includes the contact portion 462 with which the protrusion 352 of the electrode member (first electrode) 344a can be brought into contact. The contact portion 462 faces the protrusion 352. In the present embodiment, the contact portion 462 intersects (is perpendicular or substantially perpendicular to) the opening and closing directions of the jaw 404.

The base 414 includes a pair of inclined surfaces (insulating surfaces) 464a and 464b separated from each other. The inclined surface 464a is located closer to the first side surface 384a of the treatment portion 326 along the first width direction W1 than an electrode surface 452 described later. The inclined surface 464a and the electrode surface 452 may be parallel to each other or may be inclined with respect to each other. The inclined surface 464b is located closer to the second side surface 384b of the treatment portion 326 along the second width direction W2 than an electrode surface 454 to be described later. The inclined surface 464b and the electrode surface 454 may be parallel to each other or may be inclined with respect to each other.

The electrode member (second electrode) 412 includes a pair of electrode surfaces (inclined surfaces) 452 and 454 spaced apart from each other. The electrode surfaces 452 and 454 face a side where the first grasping piece 26a is located, that is, a side where the second grasping piece 26b is closed. The electrode surfaces 452 and 454 are electrically connected and have the same potential.

A virtual plane VP perpendicular to the opening and closing directions and passing through a first proximity edge 482a of the first electrode surface 452 in the closed state is defined on the first electrode surface 452 of the electrode member 412. The virtual plane VP preferably lies on the longitudinal axes L1 and L2 in the closed state. That is, in the present embodiment, the virtual plane VP is defined as a plane that is perpendicular to the motion surface T, extends along the longitudinal axes L1 and L2, and passes through the proximity edge 482a of the first electrode surface 452. With respect to the second electrode surface 454 of the electrode member 412, a virtual plane that is perpendicular to the opening and closing directions and passes through the second proximity edge 484a of the second electrode surface 454 in the closed state coincides with the above-described virtual plane VP.

The base 414 is attached to the back surfaces of the electrode surfaces 452 and 454, that is, the end surfaces of the electrode surfaces 452 and 454 on a side opposite to those where the blade 344 is located. The electrode surfaces 452 and 454 are disposed with the contact portion 462 interposed between the electrode surfaces 452 and 454, and are disposed apart from each other in the width direction of the jaw 404. Therefore, the electrode surface 452 is adjacent to one side of the contact portion 462 in the width direction of the jaw 404. The electrode surface 454 is adjacent to the other side of the contact portion 462 in the width direction of the jaw 404. Each of the contact portion 462 and the electrode surfaces 452 and 454 continuously extends over the range from the proximal end portion to the distal end portion of the jaw 404 in the longitudinal direction of the treatment portion 326. Then, when the first grasping piece 26a and the second grasping piece 26b are closed with respect to each other in a state in which no tissue is present between the first grasping piece 26a and the second grasping piece 26b, the protrusion 352 is in contact with the contact portion 462 over the range from the proximal end portion to the distal end portion of the treatment portion 326.

The first electrode surface 452 is on the inner side of the first side surface 384a of the treatment portion 326 in the width direction. The first electrode surface 452 protrudes to a side where the first grasping piece 26a is located, that is, a side where the second grasping piece 26b is closed. Similarly, the second electrode surface 454 is on the inner side of the second side surface 384b of the treatment portion 326 in the width direction. The second electrode surface 454 protrudes to a side where the first grasping piece 26a is located, that is, a side where the second grasping piece 26b is closed.

The first electrode surface (first surface) 452 is separated from the protrusion (facing surface) 352 of the blade 344 in both the opened and closed states. The second electrode surface (second surface) 454 is separated from the facing surface 352 of the blade 344 in both the opened state and the closed state.

The distance between the first outer edge 482b and the virtual plane VP is larger than the distance between the first proximity edge 482a of the first electrode surface (first surface) 452 and the virtual plane VP. In the example shown in FIG. 13 to FIG. 15, the distance between the first electrode surface 452 and the virtual plane VP continuously increases from the first proximity edge 482a toward the first outer edge 482b.

Similarly, the distance between the second outer edge 484b and the virtual plane VP is larger than the distance between the first proximity edge 484a of the second electrode surface (second surface) 454 and the virtual plane VP. In the example shown in FIG. 13 to FIG. 15, the distance between the second electrode surface 452 and the virtual plane VP continuously increases from the second proximity edge 484a toward the second outer edge 484b.

The base 414 of the second grasping piece 26b includes, between the electrode surfaces 452 and 454, the contact portion 462 facing the protrusion 352 of the blade 344. Therefore, the blade 402 of the second grasping piece 26b includes the contact portion 462, the first electrode surface (first surface) 452, the second electrode surface (second surface) 454, and a pair of inclined surfaces 464a and 464b as surfaces facing the protrusion 352, and a pair of facing surfaces 348a and 348b of the first grasping piece 26a.

The contact portion 462 has an electrical insulating property. The contact portion 462 faces the protrusion (treatment surface) 352 of the blade 344. The contact portion 462 is movable relative to the protrusion 352 of the blade 344 along the opening and closing directions between an opened state in which the contact portion is separated from the protrusion and a closed state in which the contact portion is close to the protrusion. In particular, the contact portion 462 can come into contact with the protrusion 352 of the blade 344 in the closed state. That is, in the closed state, the central surface (protrusion) 352b of the protrusion 352 of the blade 344 of the first grasping piece 26a is also located between the electrode surfaces 452 and 454 in the width direction. Therefore, a motion surface T (central position M) in the width direction of the second grasping piece 26b passes through the contact portion 462 and the central surface 352b of the protrusion 352 of the blade 344.

The contact portion 462 includes a first edge portion 472a separated from the central position M in the first width direction W1, a second edge portion 472b separated from the central position M in the second width direction W2, and a central portion 474 through which the motion surface T passes. The first edge portion 472a is closer to the first side surface 384a of the treatment portion 326 than to the second side surface 384b of the treatment portion 326. Therefore, the first edge portion 472a of the contact portion 462 is brought in proximity to the first side surface 384a of the treatment portion 26 which is separated from the longitudinal axis L2 of the contact portion 462 in the first width direction W1 perpendicular to the opening and closing directions. The second edge portion 472b is closer to the second side surface 384b of the treatment portion 326 than to the first side surface 384a of the treatment portion 326. Therefore, the second edge portion 472b of the contact portion 462 is close to the second side surface 384b of the treatment portion 326 which is separated from the longitudinal axis L2 of the contact portion 462 in the second width direction W2 perpendicular to the opening and closing directions.

The central portion 474 is formed between the first edge portion 472a and the second edge portion 472b. Here, the central portion 474 is formed in a flat shape with which the vicinity of the central portion of the protrusion 352 of the blade 344 of the first grasping piece 26a is brought into contact. Therefore, in the present embodiment, the three surfaces 352a, 352b, and 352c of the protrusion 352 are brought into contact with the central portion 474 of the contact portion 462. Of course, only the central surface 352b may be in contact with the central portion 474 of the contact portion 462.

The first electrode surface 452 includes a first proximity edge 482a that is in proximity to the longitudinal axis L2 and a first outer edge 482b spaced farther from the longitudinal axis L2 than the first proximity edge 482a and in proximity to the first side surface 384a of the treatment portion 326.

It is preferable that there be no clearance between the first edge portion 472a of the contact portion 462 and the proximity edge 482a of the first electrode surface 452, but there may be a slight clearance between the first edge portion 472a and the proximity edge 482a. In addition, it is preferable that there be no step between the first edge portion 472a of the contact portion 462 and the proximity edge 482a of the first electrode surface 452, but there may be a slight step between the first edge portion 472a and the proximity edge 482a. The first electrode surface 452 is provided from the first edge portion 472a of the contact portion 462 or the first proximity edge 482a of the first electrode surface 452 toward the first side surface 384a of the treatment portion 26. In the present embodiment, the first electrode surface (first surface) 452 is formed in a flat shape between the first edge portion 472a of the contact portion 462 or the proximity edge 482a of the first electrode surface 452 and the outer edge 482b of the first electrode surface 452.

In one embodiment, the area between the proximity edge 482a of the first electrode surface 452 and the outer edge 482b of the first electrode surface 452 may be a non-planar, combination curved surface/planar surface, similar to the example of the electrode surface 152 described in FIG. 5A to FIG. 7. That is, the first electrode surface 452 may be a flat surface or a curved surface. Furthermore, the first electrode surface 452 may be formed of one or more flat surfaces and one or more curved surfaces. In addition, the distance between the first electrode surface 152 and the virtual plane VP may continuously or intermittently increase from the first proximity edge 182a toward the first outer edge 182b.

The inclined surface 464a is formed between the first outer edge 482b of the electrode surface 452 and the first side surface 384a of the treatment portion 326. The inclined surface 464b is formed between the second outer edge 484b of the electrode surface 454 and the second side surface 384b of the treatment portion 326. In FIG. 13 to FIG. 15, the first outer edge 482b of the electrode surface 452 is located at a position protruding from the inclined surface 464a toward the first grasping piece 26a. However, the first outer edge 482b of the electrode surface 452 and the end portion 465a of the inclined surface 464a close to the longitudinal axis L2 may be flush with each other. The second outer edge 484b of the electrode surface 454 is located at a position protruding from the inclined surface 464b toward the first grasping piece 26a. However, the second outer edge 484b of the electrode surface 454 and the end portion 465b of the inclined surface 464b close to the longitudinal axis L2 may be flush with each other.

The first electrode surface 452 is inclined in the width direction so as to separate from the side where the first grasping piece 26a is located as the distance to the first side surface 384a of the treatment portion 326 is reduced. In other words, the electrode surface 452 is directed to the side where the distance from the first grasping piece 26a increases as the electrode surface separates from the central position M in the first width direction W1.

Here, the contact portion 462 of the base 414 of the blade 402 of the second grasping piece 26b can be brought into contact with the protrusion 352 of the blade 344 of the first grasping piece 26a in the closed state. A virtual plane VP perpendicular to the opening and closing directions and passing through the first proximity edge 482a of the first electrode surface 452 in the closed state is defined on the first electrode surface 452 of the electrode member 412. The virtual plane VP preferably lies on the longitudinal axes L1 and L2 in the closed state. That is, in the present embodiment, the virtual plane VP is defined as a plane that is perpendicular to the motion surface T, extends along the longitudinal axes L1 and L2, and passes through the proximity edge 482a of the first electrode surface 452. A virtual plane, passing through the second proximity edge 484a of the second electrode surface 454 and defined between the protrusion 352 and the second proximity edge 484a of the second electrode surface 454 on the second side surface 384b side of the treatment portion 326 separated from the longitudinal axes L1 and L2 in a second width direction W2 perpendicular to the opening and closing directions, coincides with the above-described virtual plane VP.

The distance between the first electrode surface 452 and the virtual plane VP increases (becomes larger) as the virtual point moves away from the longitudinal axis L2 toward the first side surface 384a of the treatment portion 326, in a range between the first edge portion 472a of the contact portion 462 or the proximity edge 482a of the first electrode surface 452 and the outer edge 482b of the first electrode surface 452. A distance Ld at a position separated from the first proximity edge 482a is larger than a distance Lp between the virtual plane VP and the first electrode surface 452 at a position close to the first proximity edge 482a in FIG. 13.

A normal vector N2a on the first electrode surface (first surface) 452 is considered. Of the normal vector N2a of the first electrode surface 452, components parallel to the virtual plane VP are directed to the first side surface 384a of the treatment portion 326 at any position of the first electrode surface 452.

The second electrode surface 454 is provided from the second edge portion 472b of the contact portion 462 toward the second side surface 384b of the treatment portion 326.

The second electrode surface 454 includes the second proximity edge 484a that is in proximity to the longitudinal axis L2, and the second outer edge 484b spaced farther from the longitudinal axis L2 than the second proximity edge 484a and in proximity to the second side surface 384b of the treatment portion 326.

In the present embodiment, the first electrode surface 452 and the second electrode surface 454 are symmetrical with respect to the motion surface T (central position M).

A normal vector N2b on the second electrode surface (second surface) 454 is considered. Of the normal vector N2b of the second electrode surface 454, components parallel to the virtual plane VP are directed to the second side surface 384b of the treatment portion 326 at any position of the second electrode surface 454.

A normal vector N1a on the first proximity surface 352a of the facing surface 352 of the blade 344 is considered. Of the normal vector N1a of the first electrode surface 352a, components parallel to the virtual plane VP are directed to the first side surface 384a of the treatment portion 326 at any position of the first proximity surface 352a. A normal vector N1b on the second proximity surface 352c of the facing surface 352 of the blade 344 is considered. Of the normal vector N1b of the second proximity surface 352c, components parallel to the virtual plane VP are directed to the second side surface 384b of the treatment portion 226 at any position of the second proximity surface 352c.

As the blade 402 of the second grasping piece 26b, the electrode surface 452, the contact portion 462, and the electrode surface 454 may be formed at an acute angle, a right angle, or an obtuse angle as a whole. The width between the first edge portion 472a and the second edge portion 472b of the contact portion 462 is preferably as small as possible. Therefore, it is preferable that the blade 402 of the second grasping piece 26b be formed to be as sharp as possible.

(Operation)

Next, an operation of the treatment instrument 312 according to the present embodiment will be described. Descriptions of the parts identical to those of the first embodiment will be appropriately omitted.

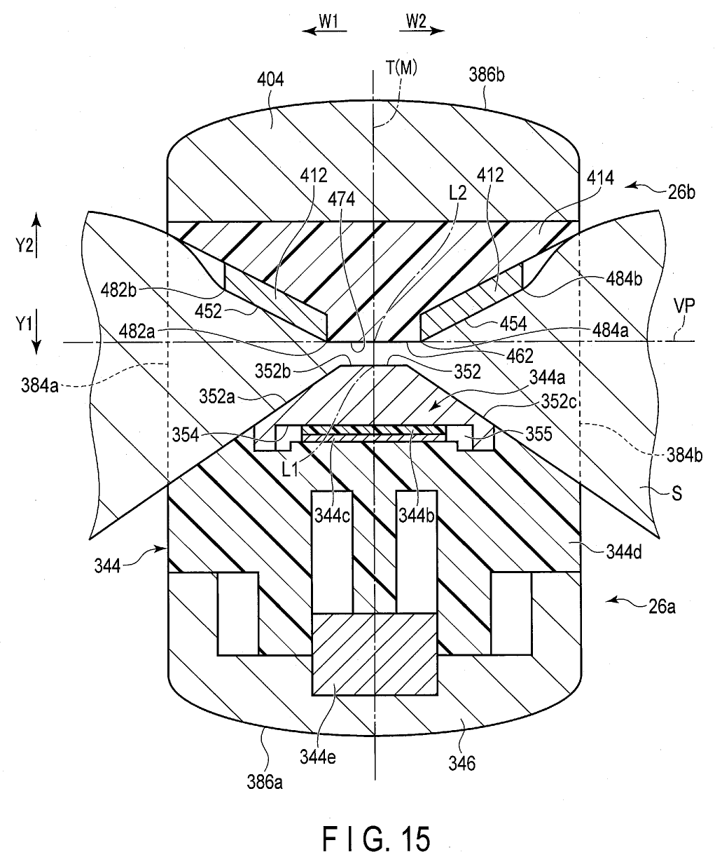

When performing a treatment using the treatment instrument 312, an operator inserts the treatment portion 326 into a body cavity such as an abdominal cavity. Then, a treatment target S such as a living tissue (for example, a blood vessel) is placed between the first grasping piece 26a and the second grasping piece 26b, and the treatment target S is grasped between the first grasping piece 26a and the second grasping piece 26b (see FIG. 15).

An appropriate gripping pressure is applied between the protrusion 352 of the electrode member 344a of the blade 344 of the first grasping piece 26a and the contact portion 462 of the blade 402 of the second grasping piece 26b. When the first grasping piece 26a and the second grasping piece 26b are closed, the treatment target is thinned by the grasping pressure on the motion surface T. Therefore, when the treatment instrument 312 according to the present embodiment is used, an appropriate gripping pressure is applied to the treatment target between the blade 344 of the first grasping piece 26a and the contact portion 462 of the second grasping piece 26b, instead of between electrodes. Further, the treatment target is brought into contact with the protrusion 352 of the electrode member 344a of the blade 344 of the first grasping piece 26a, and the treatment target is brought into contact with the first electrode surface 452 and the second electrode surface 454 of the electrode member 412 of the second grasping piece 26b.

In this state, the operator presses, for example, a first pedal of the operating device 318 that functions similarly to the first switch 16a of the first embodiment described above. The energy source 16 outputs electrical energy based on an operation input at the first pedal of the operating device 318.

The electrode member 344a of the blade 344 of the first grasping piece 26a and the electrode member 412 of the second grasping piece 26b function as electrodes having different potentials with respect to each other. A high-frequency current is passed through the treatment target grasped between the electrode member 344a of the blade 344 of the first grasping piece 26a and the electrode member 412 of the second grasping piece 26b, and the high-frequency current is applied to the treatment target S as treatment energy. The heat generated due to the high-frequency current denatures the treatment target and promotes the coagulation of the treatment target. That is, the blood vessel or the like, which is the treatment target, is gelatinized, joined, and sealed by the heat generated due to the high-frequency current. Thus, the treatment instrument 312 can seal (treat) the treatment target.

In the treatment portion 326 of the treatment instrument 312 according to the present embodiment, in particular, by forming the blade 402 of the second grasping piece 26b as described above, it is possible to efficiently increase the temperature of the treatment target and to suppress the temperature increase of the blade 344 of the first grasping piece 26a in the same manner as in the first embodiment described above. In addition, in the same manner as in the first embodiment described above, when the treatment instrument 312 according to the present embodiment is used, since it is possible to suppress the temperature increase of the blade 344 of the first grasping piece 26a, energy is efficiently applied to the blood vessel as the treatment target S. Therefore, in the case of using the treatment instrument 312 according to the present embodiment, the sealing can be performed at a higher speed compared to the case of using the conventional treatment instrument.

Next, an example will be described in which a second pedal of the operating device 318 that functions in the same manner as the second switch 16b of the first embodiment described above is pressed. Based on the operation input at the second pedal of the operating device 318, the energy source 16 outputs electrical energy to the treatment instrument 312.

When electrical energy is supplied from the energy source 16 to the heat generating portion 344c, the heat generating portion 344c generates heat. The heat generated in the heat generating portion 344c is transferred from the back surface side to the protrusion 352 in the electrode member 344a via the adhesive layer 344b. The temperature of the heat transferred to the protrusion 352 is set higher than the temperature of the treatment target that can be increased by the high-frequency current. The heat (heat energy) transferred to the protrusion 352 of the blade 44 of the first grasping piece 26a is applied to the treatment target. At this time, the treatment target is coagulated and incised between the protrusion (facing surface) 352 of the blade 344 and the contact portion 462 of the blade 402.

As described above, the treatment target is thinned by the protrusion 352 of the blade 344 of the first grasping piece 26a and the contact portion 462 of the blade 402 of the second grasping piece 26b. Therefore, the treatment target S efficiently increases in temperature. Accordingly, drying of the treatment target S easily proceeds, and the speed of incising the treatment target S is improved. When the treatment target is incised, the treatment target is coagulated in a portion closer to the first side surface 384a and a portion closer to the second side surface 384b of the treatment portion 326 than the incision position of the treatment target.

Therefore, the treatment portion 326 of the treatment instrument 312 according to the present embodiment incises the treatment target more easily than the treatment portion of the conventional treatment instrument. Therefore, when heat is transferred to the treatment target, the treatment portion 326 of the treatment instrument 312 according to the present embodiment is likely to incise the treatment target with less energy than that required by the treatment portion of the conventional treatment instrument.

When the second pedal of the operating device 318 is pressed, the system 10 may cause a high-frequency current to flow through the treatment target to coagulate and incise the treatment target S at the same time when the temperature of the heat generating portion 344c is increased, in the same manner as in the time of pressing the first pedal of the operating device 318.

According to the present embodiment, in the case where the incision treatment is performed by the energy that is different from the high-frequency energy and generated by the heat generating portion 344c of the first grasping piece 26a, the treatment instrument 312 capable of efficiently applying the energy to the treatment target S can be provided by forming the blade 402 of the second grasping piece 26b in an appropriate shape. Therefore, by using the treatment instrument 312 according to the present embodiment, the incision treatment can be efficiently performed on the treatment target with less energy (amount). That is, the blade 402 of the second grasping piece 26b can incise (treat) the treatment target in cooperation with the protrusion (treatment surface) 352 of the blade 344 of the first grasping piece 26a.

Furthermore, in a case where the treatment target S is to be coagulated by using the high-frequency energy, it is possible to provide the treatment instrument 312 capable of suppressing the temperature increase of the electrode (the electrode member 344a of the blade 344) when the high-frequency current flows, and efficiently applying the energy to the treatment target S. Thus, the treatment instrument 312 can coagulate (treat) the treatment target.

In the present embodiment described above, as an example, the second grasping piece 26b is movable with respect to the first grasping piece 26a fixed to the distal end of the relay portion 328. It is also preferable that both the first grasping piece 26a and the second grasping piece 26b be movable with respect to the distal end of the relay portion 328.

### First Modification

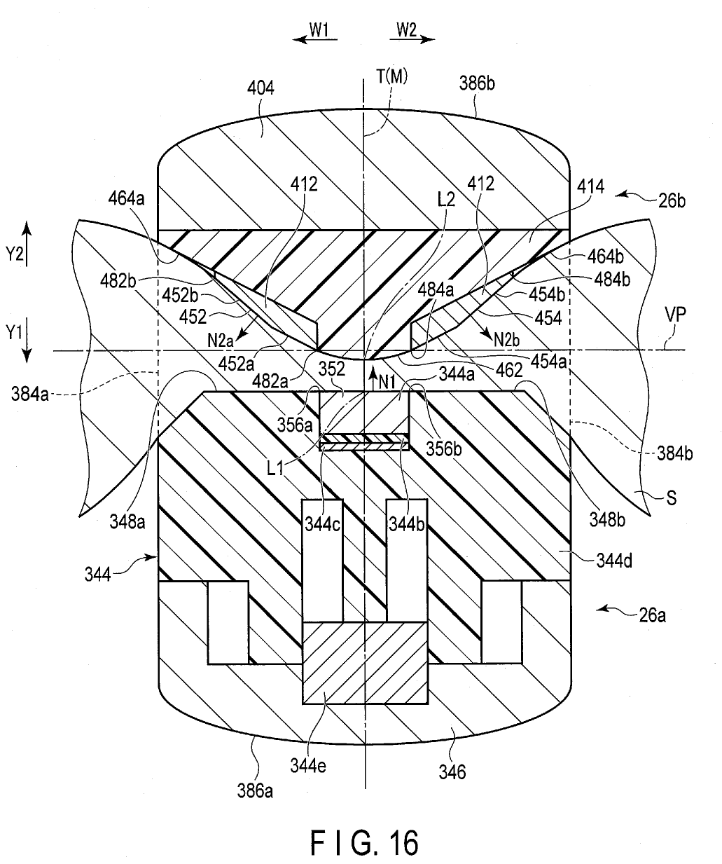

Next, a first modification of the second embodiment will be described with reference to FIG. 16. This modification is a modification of the first embodiment including its modifications and/or the second embodiment, and the same members or members having the same functions as those described above are denoted by the same reference numerals as much as possible, and detailed descriptions thereof will be omitted.

The contact portion 462 of the second grasping piece 26b shown in FIG. 13 to FIG. 15 is, for example, planar. The contact portion 462 shown in FIG. 16 includes a curved surface projecting toward the protrusion 352 of the first grasping piece 26a. Therefore, in the closed state, the contact portion 462 is at the same level as the first electrode surface 452 or protrudes toward the treatment surface 352 from the first electrode surface 452. Even when the protrusion 352 includes a curved surface, the treatment is performed in the same manner as in the second embodiment described above.

The contact portion 462 of the second grasping piece 26b may be flat as shown in the second embodiment or may be concave as shown in the first embodiment.

The facing surfaces 348a and 348b of the first grasping piece 26a have a combination of a region parallel to the virtual plane VP and a region inclined with respect to the virtual plane VP. The facing surfaces 348a and 348b may be formed only by surfaces parallel to the virtual plane VP.

A normal vector N1 on the facing surface 352 of the blade 344 is considered. Of the normal vector N1, components parallel to the virtual plane VP do not include components toward the first side surface 384a and components toward the second side surface 384b at any position of the facing surface 352. In the example shown in FIG. 16, the facing surfaces 348a and 348b of the base 344d are continuous with the facing surface 352 of the blade 344. Of the facing surfaces 348a and 348b, positions close to the facing surface 352 of the blade 344 are parallel to the virtual plane VP. Of the facing surfaces 348a and 348b, positions separated from the facing surface 352 of the blade 344 are inclined to the virtual plane VP. At any position of the facing surface 348a of the base 344d, there may be components of the normal vectors N1 directed toward the first side surface 384a, but there are no components directed toward the second side surface 384b. At any position of the facing surface 348b of the base 344d, there may be components of the normal vectors N1 directed toward the second side surface 384b, but there are no components directed toward the first side surface 384a.

The first electrode surface 452 is inclined in the width direction so as to separate from the side where the first grasping piece 26a is located as the distance to the first side surface 384a of the treatment portion 326 is reduced. In other words, the electrode surface 452 is directed to the side where the distance from the first grasping piece 26a increases as the electrode surface separates from the central position M in the first width direction W1. Here, the first electrode surface (first surface) 452 includes a first region 452a including the proximity edge 482a and a second region 452b including the outer edge 482b along the width direction. The first region 452a and the second region 452b are each formed as a flat surface. Thus, the first electrode surface 452 is formed by a plurality of flat surfaces. These regions 452a and 452b are inclined similarly to the regions 186a and 186b described as the second modification (see FIG. 6A) or the third modification (see FIG. 6B) of the first embodiment. That is, the inclination angles formed by the first region 452a including the first proximity edge 482a and the second region 452b including the first outer edge 482b with respect to the virtual plane VP are different from each other.

The second electrode surface 454 is inclined in the width direction so as to separate from the side where the first grasping piece 26a is located as the distance to the second side surface 384b of the treatment portion 326 is reduced. In other words, the electrode surface 454 is directed to the side where the distance from the first grasping piece 26a increases as the electrode surface separates from the central position M in the second width direction W2. Here, the second electrode surface (second surface) 454 includes, along the width direction, a first region 454a including the proximity edge 484a and a second region 454b including the outer edge 482b. The first region 454a and the second region 454b are each formed as a flat surface. Thus, the second electrode surface 454 is formed by a plurality of flat surfaces. These regions 454a and 454b are inclined similarly to the regions 188a and 188b described as the second modification (see FIG. 6A) or the third modification (see FIG. 6B) of the first embodiment. That is, the inclination angles formed by the second region 454a including the first proximity edge 484a and the second region 454b including the second outer edge 484b with respect to the virtual plane VP are different from each other.

The first electrode surface 452 and the second electrode surface 454 each may be a flat surface or a curved surface. The first electrode surface 452 and the second electrode surface 454 each may be formed of one or more flat surfaces and one or more curved surfaces.

In FIG. 16, the first outer edge 356a of the facing surface 352 of the blade 344 is located closer to the motion surface T than the first proximity edge 482a of the first electrode surface 452 of the blade 402. As long as the facing surface 352 of the blade 344 and the first electrode surface 452 of the blade 402 are not in contact with each other, the first outer edge 356a of the facing surface 352 of the blade 344 may be located farther from the motion surface T than the first proximity edge 482a of the first electrode surface 452 of the blade 402.

### Second Modification

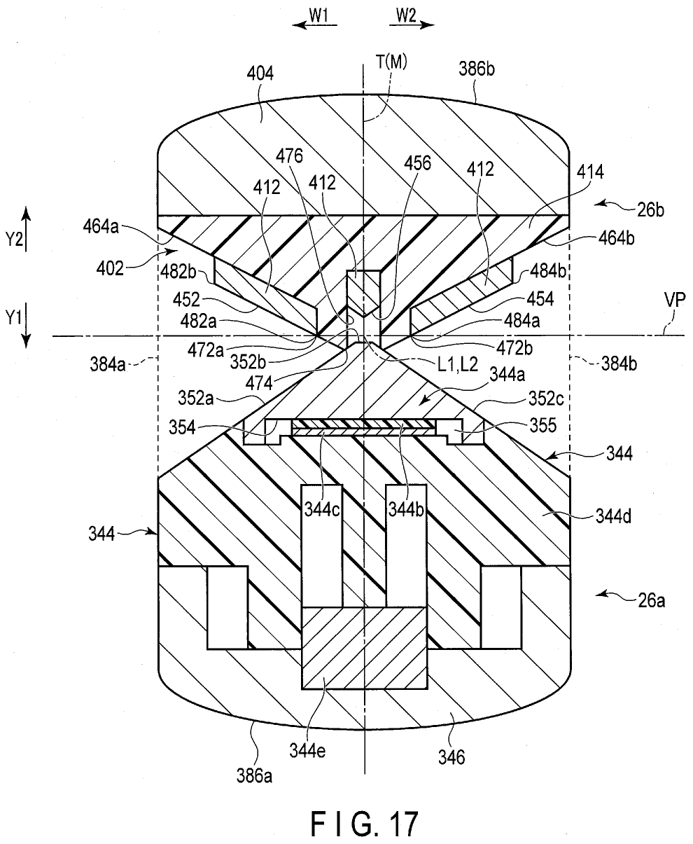

Next, a second modification of the second embodiment will be described with reference to FIG. 17. This modification is a further modification of the first embodiment including its modifications and/or the second embodiment including the first modification, and the same members or members having the same functions as those described above are denoted by the same reference numerals as much as possible, and detailed descriptions thereof will be omitted.

As shown in FIG. 17, the first edge portion 472a of the contact portion 462 may be located at an equal distance to the second back surface 386b of the treatment portion 326 from the proximity edge 482a of the second electrode surface 452 of the electrode member 412 along the opening and closing directions or may be located closer to the second back surface 386b than the proximity edge 482a.

A recess (concave portion) 476 is formed in the central portion 474 of the base 414 of the second grasping piece 26b shown in FIG. 17. That is, the contact portion 462 includes the recess 476 which is recessed in a direction opposite to a direction toward the protrusion 352 of the electrode member 344a of the first grasping piece 26a between the first edge portion 472a and the second edge portion 472b.

The blade 402 of the second grasping piece 26b includes, in the recess 476, an electrode surface (third surface) 456 used as a high-frequency electrode and having the same potential as that of the first electrode surface 452 and the second electrode surface 454.

In a state in which the treatment target is gripped between the first grasping piece 26a and the second grasping piece 26b, the treatment target may come into contact with the electrode surface 456 in the recess 476 before the high-frequency current is applied or while the high-frequency current is being applied. When the treatment target S comes into contact with the electrode surface 456, a current flows not only between the first electrode surface 452 and the protrusion 352 and between the second electrode surface 454 and the protrusion 352, but also between the electrode surface 456 and the protrusion 352.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment instrument comprising:
a first treatment body having a treatment surface to be used as a high-frequency electrode, and configured to receive, together or separately, a high-frequency energy and another energy different from high-frequency energy input to the treatment surface; and
a second treatment body configured to treat a treatment target in cooperation with the treatment surface, the second treatment body including:
a contact portion extending along a longitudinal axis and having an electrical insulating property; and
a first surface including a first high-frequency electrode,
wherein:
the first surface is adjacent to the contact portion in a first width direction perpendicular to the longitudinal axis,
the contact portion is configured to face the treatment surface of the first treatment body, the treatment instrument being configured to move between an opened state, wherein, in the open state, the contact portion is spaced apart from the treatment surface along opening and closing directions and a closed state, wherein, in the closed state, the contact portion abuts the treatment surface, and
the first surface is spaced apart from the treatment surface in both the opened state and the closed state, the first surface including:
a first proximity edge of the first surface, the first proximity edge being in proximity to the contact portion; and
a first outer edge spaced from the contact portion from the longitudinal axis toward a first side surface of a treatment portion in the first width direction, wherein:
with a virtual plane being provided as a reference, the virtual plane is perpendicular to the opening and closing directions in the closed state and intersects the first proximity edge of the first surface, a distance between the first outer edge and the virtual plane is larger than a distance between the first proximity edge and the virtual plane.

2. The treatment instrument according to claim 1, wherein the distance between the virtual plane and the first surface increases continuously or intermittently from the first proximity edge toward the first outer edge.

3. The treatment instrument according to claim 1, wherein the first surface is formed by a flat surface or a curved surface.

4. The treatment instrument according to claim 1, wherein the first surface includes a plurality of surfaces.

5. The treatment instrument according to claim 1, wherein the first surface includes one or more flat surfaces and one or more curved surfaces.

6. The treatment instrument according to claim 1, wherein a first inclination angle formed between the virtual plane and a first region including the first proximity edge and a second inclination angle formed between the virtual plane and a second region including the first outer edge are different.

7. The treatment instrument according to claim 1, wherein the first surface includes, between the first proximity edge and the first outer edge, a region in which the distance between the virtual plane and the first surface is constant.

8. The treatment instrument according to claim 1, wherein, in the closed state, the contact portion is level with the first surface or protrudes from the first surface toward the treatment surface.

9. The treatment instrument according to claim 1, wherein in the closed state, the first outer edge of the first surface is spaced farther from the longitudinal axis than an outer edge in the treatment surface of the first treatment body.

10. The treatment instrument according to claim 1, wherein a cross section of a part of the first surface between the first proximity edge and the first outer edge is formed in a linear state.

11. The treatment instrument according to claim 1, wherein a cross section of a part of the first surface between the first proximity edge and the first outer edge is formed in a nonlinear state.

12. The treatment instrument according to claim 1, wherein:
the second treatment body includes a second surface and is configured to be a second high-frequency electrode having a same potential as the first high-frequency electrode;
the second surface is adjacent to the contact portion in a second width direction opposite to the first width direction; and
the second surface is spaced apart from the treatment surface in the opened state and the closed state, and includes:
a second proximity edge of the second surface, the second proximity edge being in proximity to the contact portion; and
a second outer edge spaced from the contact portion from the longitudinal axis toward a second side surface of the treatment portion in the second width direction, and
wherein:
the virtual plane passes through the second proximity edge of the second surface, and
a distance between the second outer edge and the virtual plane is larger than a distance between the second proximity edge and the virtual plane.

13. The treatment instrument according to claim 12, wherein the second surface includes a region in which the distance between the virtual plane and the second surface is constant between the second proximity edge and the second outer edge.

14. The treatment instrument according to claim 1, wherein:
the contact portion includes a first edge portion that is adjacent to the first proximity edge of the first surface, a second edge portion that is spaced apart from the first proximity edge, and a recess that is provided between the first edge portion and the second edge portion and that is recessed in a direction that is opposite to the direction toward the treatment surface; and
the second treatment body includes, in the recess, a third surface to be used as a high-frequency electrode having a same potential as the first surface.

15. The treatment instrument according to claim 1, wherein:
the treatment surface of the first treatment body is a part of a rod,
when ultrasonic vibrations are input to the rod as another energy different from the high-frequency energy, the ultrasonic vibrations are configured to be transmitted to the treatment surface of the first treatment body, and
the treatment instrument comprising a shaft through which the rod is inserted, wherein the first treatment body is fixed relative to the shaft, and the second treatment body is configured to move relative to the shaft.

16. The treatment instrument according to claim 1, wherein the first treatment body includes a heat generating portion configured to input heat to the treatment surface as another energy different from the high-frequency energy.

17. The treatment instrument according to claim 1, comprising a first jaw provided with the first treatment body, configured to move together with the treatment surface relative to the second treatment body in the opened state and the closed state, and spaced from the contact portion and the first surface of the second treatment body in the closed state.

18. The treatment instrument according to claim 1, comprising a second jaw provided with the second treatment body, movable together with the contact portion and the first surface relative to the first treatment body in the opened state and the closed state, and spaced from the treatment surface of the first treatment body in the closed state.

19. A treatment system comprising:
the treatment instrument according to claim 1, and
an energy source configured to supply an electrical energy to the treatment instrument.

20. A treatment instrument comprising:
a first treatment body including a first treatment surface having an electrical conductivity, and configured to input energy thereto;
a second treatment body configured to treat a treatment target in cooperation with the first treatment surface, the second treatment body including:
a contact portion extending along a longitudinal axis and having an electrically insulating property; and
a second treatment surface having an electrical conductivity, and configured to input energy thereto,
wherein the second treatment surface is adjacent to the contact portion in a width direction perpendicular to the longitudinal axis,
the contact portion is configured to face the first treatment surface of the first treatment body, the treatment instrument being configured to move between an opened state in which the contact portion is spaced apart from the first treatment surface along opening and closing directions and a closed state in which the contact portion abuts the first treatment surface, and
the second treatment surface is spaced apart from the first treatment surface in the opened state and the closed state, and includes
a proximity edge of the second treatment surface, the proximity edge being in contact with the contact portion; and
an outer edge spaced from the contact portion from the longitudinal axis toward a side surface of a treatment portion in the width direction, wherein:
a virtual plane is provided as a reference and defined as being perpendicular to the opening and closing directions in the closed state and passing through the proximity edge of the second treatment surface, a distance between the outer edge and the virtual plane is larger than a distance between the proximity edge and the virtual plane.
